(19) United States
(12) Patent Application Publication
(10) Pub. No.: US 2026/0215967 A1
(43) Pub. Date: Jul. 30, 2026

(54) SHUNTING SYSTEMS WITH VISUAL STATE INDICATORS AND/OR FLOW INDICATORS

(71) Applicant: Shifamed Holdings, LLC, Campbell, CA (US)

(72) Inventors: David Batten, San Francisco, CA (US); Tessa Bronez, Campbell, CA (US); Tom Saul, Portland, OR (US); Richard Lilly, San Jose, CA (US); Eric Schultz, Los Altos, CA (US); Abigail Brazil, San Francisco, CA (US); Amr Salahieh, Saratoga, CA (US)

(21) Appl. No.: 19/148,576

(22) PCT Filed: Jan. 26, 2024
(86) PCT No.: PCT/US2024/013197

§ 371 Date: Jul. 16, 2025

Related U.S. Application Data
(60) Provisional application No. 63/481,955, filed on Jan. 27, 2023.
(60) Provisional application No. 63/578,697, filed on Aug. 25, 2023.
(60) Provisional application No. 63/610,578, filed on Dec. 15, 2023.

(51) Int. Cl. A61F 9/007; A61B 17/00

(52) U.S. Cl. CPC A61F 9/00781; A61B 2017/00017; A61B 2017/00367; A61F 2250/0097

(57) ABSTRACT

The present technology is generally directed to shunting systems having visual system state indicators and/or flow indicators. The system state indicators assist a user in determining a state of the shunt, such as whether a shunt lumen is set to an open or closed position. The flow indicators assist a user in determining whether fluid is flowing through the shunt lumen.

### CROSS-REFERENCE TO RELATED APPLICATION(S)

This application claims priority to U.S. Provisional Patent Application No. 63/481,955, filed Jan. 27, 2023, U.S. Provisional Patent Application No. 63,578,697, filed Aug. 25, 2023, and U.S. Provisional Patent Application No. 63/610,578, filed Dec. 15, 2023, each of which is incorporated by reference herein in its entirety.

### TECHNICAL FIELD

The present technology generally relates to implantable medical devices and, in particular, to shunting systems for promoting fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma shunts or “MIGS”) have shown clinical benefit; however, there is a need for improved shunting systems and techniques for addressing elevated intraocular pressure and risks associated with glaucoma, as well as other patient conditions. For example, there is a need for shunting systems capable of adjusting the therapy provided, including the flow rate/fluid resistance between the two fluidly-connected bodies. As another example, there is a need for a shunting system capable of being modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates an adjustable shunting system configured in accordance with select embodiments of the present technology.

FIG. 1B is an exploded view of the adjustable shunting system of FIG. 1A.

FIG. 1C is an enlarged exploded view of an actuation assembly of the adjustable shunting system of FIGS. 1A and 1B.

FIG. 1D is an enlarged view of an actuator of the adjustable shunting system shown in FIGS. 1A-1C.

FIG. 2A is an enlarged view of a plate of the adjustable shunting system shown in FIGS. 1A-1C and including various system state indicators configured in accordance with select embodiments of the present technology.

FIGS. 2B and 2C are enlarged, cut-away views of select portions of the plate shown in FIG. 2A.

FIG. 3A is a top view of another adjustable shunting system configured in accordance with select embodiments of the present technology.

FIG. 3B is an enlarged top view of a portion of the adjustable shunting system of FIG. 3A that includes an actuation assembly.

FIGS. 4A-4F illustrate various system state indicators for use with an adjustable shunting system and configured in accordance with select embodiments of the present technology.

FIG. 5 is a schematic illustration of a system state indicator for use with an adjustable shunting system and configured in accordance with select embodiments of the present technology.

FIGS. 6A and 6B illustrate another system state indicator for use with an adjustable shunting system and configured in accordance with select embodiments of the present technology.

FIG. 7 illustrates a first embodiment of a flow indicator assembly for detecting fluid flow through a shunt and configured in accordance with select embodiments of the present technology.

FIG. 8 illustrates a second embodiment of a flow indicator assembly for detecting fluid flow through a shunt and configured in accordance with select embodiments of the present technology.

FIG. 9 illustrates a third embodiment of a flow indicator assembly for detecting fluid flow through a shunt and configured in accordance with select embodiments of the present technology.

FIG. 10 illustrates a fourth embodiment of a flow indicator assembly for detecting fluid flow through a shunt and configured in accordance with select embodiments of the present technology.

FIG. 11 illustrates a fifth embodiment of a flow indicator assembly for detecting fluid flow through a shunt and configured in accordance with select embodiments of the present technology.

FIG. 12 illustrates a sixth embodiment of a flow indicator assembly for detecting fluid flow through a shunt and configured in accordance with select embodiments of the present technology.

FIGS. 13A and 13B illustrate a seventh embodiment of a flow indicator assembly for detecting fluid flow through a shunt and configured in accordance with select embodiments of the present technology.

FIGS. 14A and 14B illustrate the adjustable shunting system of FIGS. 1A and 1B with the flow indicator assembly of FIGS. 13A and 13B and configured in accordance with select embodiments of the present technology.

### DETAILED DESCRIPTION

The present technology is generally directed to shunting systems for promoting the flow of fluid between a first body region and a second body region of a patient. As described throughout this Detailed Description, the shunting systems may include one or more visual indicators for providing a physician or other user with visual feedback regarding the performance of the shunt. For example, in some embodiments the shunting systems include system state indicators. Representative system state indicators include, but are not limited to, indicators of (1) a position of an adjustable element (e.g., an actuator) of the shunt, (2) a state of a shunt lumen (e.g., open to flow or closed to flow), and/or (3) actuation targets for adjusting a position of the adjustable element and/or the state of the shunt lumen. In addition to or in lieu of the system state indicators, in some embodiments the shunting systems include flow indicators that enable a physician or other user to visually determine, post-implantation and in real time, whether fluid is flowing through the shunt. Without intending to be bound by theory, incorporating system state indicators and/or flow indicators into shunting systems is expected to assist a physician or other user in quickly and accurately identifying a state of a shunt and evaluating performance of the shunt to ensure the patient is receiving adequate therapy.

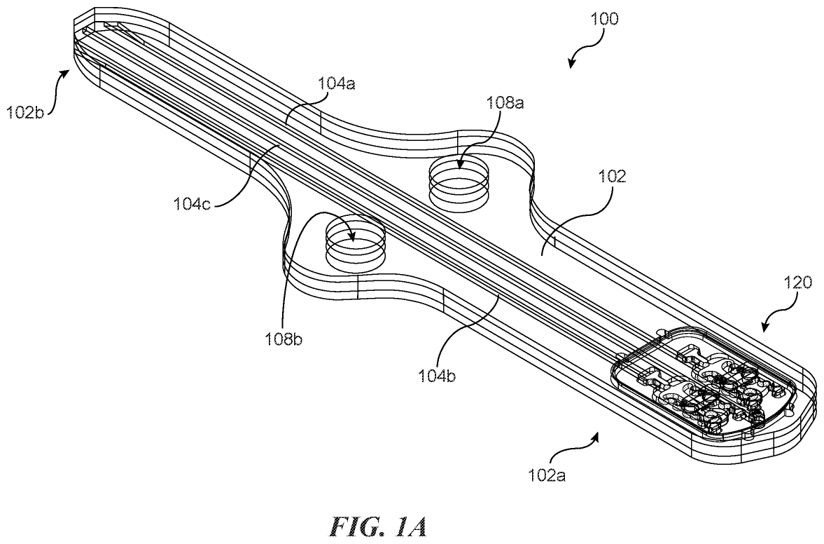

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and claims but are not described in detail with respect to FIGS. 1-14B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

The systems described herein can be designed for shunting fluid between a variety of body regions. For example, many of the embodiments described herein are designed to be implanted in a patient's eye to shunt aqueous between the anterior chamber and a target outflow location (e.g., a subconjunctival bleb space), such as to treat glaucoma. However, although certain embodiments are described in terms of shunting fluid from an anterior chamber of an eye, one of skill in the art will appreciate that the present technology can be readily adapted to shunt fluid from and/or between other portions of the eye or, more generally, from and/or between a first body region and a second, different body region of a patient. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

The headings below are provided by way of convenience only and are not to be used to interpret the scope of the claimed technology.

### A. Select Embodiments of Adjustable Shunting Systems

FIGS. 1A-1D illustrate an adjustable shunting system 100 (“the system 100”) configured in accordance with select embodiments of the present technology. More specifically, FIG. 1A is a perspective view of the system 100, FIG. 1B is an exploded view of the system 100, FIG. 1C is an enlarged view of an actuation assembly 120 of the system 100 as shown in FIG. 1B, and FIG. 1D is an enlarged view of a first actuator 124a of the actuation assembly 120. As described in greater detail below, the system 100 is configured to provide a titratable therapy for shunting fluid from a first body region to a second body region, such as shunting aqueous from an anterior chamber of a patient's eye to a target outflow location.

Referring first to FIG. 1A, the system 100 includes a shunting element 102 and an actuation assembly 120. The shunting element 102 (which can also be referred to as an elongated housing) extends between a first end portion 102a and a second end portion 102b. A plurality of flow channels 104 (shown as a first channel 104a, a second channel 104b, and a third channel 104c) can extend through the shunting element 102 at least partially between the first end portion 102a and the second end portion 102b. The channels 104 can be fluidly isolated along a portion or substantial portion of the length of the shunting element 102. As described in greater detail below, when the system 100 is implanted within a patient between a first body region and a second body region, fluid can flow from the first body region to the second body region via the channels 104. The shunting element 102 may optionally include one or more features to facilitate anchoring the system 100 to patient tissue, such as first and second suture holes 108a, 108b. The shunting element 102 can be composed of a partially flexible and/or biocompatible material, such as silicone, polydimethylsiloxane (PDMS), polymethylmethacrylate (PMA), or the like. For example, the shunting element 102 may be composed of a material having a durometer of between about 60 and about 90, or between about 70 and 80, or about 75. Additional features of shunting elements suitable for use with the present technology are described in International Patent Application No. PCT/US2022/037747, the disclosure of which is incorporated by reference herein in its entirety and for all purposes.

The actuation assembly 120 can be positioned at the first end portion 102a of the shunting element 102. As described in greater detail below, the actuation assembly 120 can have one or more features that selectively control the flow of fluid through one or more of the channels 104. In this way, the actuation assembly 120 can be selectively manipulated by a user to adjust the resistance through the system 100, and thus the level of therapy provided by the system 100.

Referring next to FIG. 1B, the shunting element 102 can one or more components and/or layers that are stacked and sealed together to collectively form the shunting element 102. For example, the shunting element 102 can include a first (e.g., top) layer 110, a second (e.g., middle) layer 112, and a third (e.g., bottom) layer 114. Accordingly, in the illustrated embodiment the shunting element 102 includes three layers, although in other embodiments the shunting element 102 can include more or fewer layers, such as one, two, four, five, six, or more layers. In operation, the first layer 110, the second layer 112, and the third layer 114 are sealed together (e.g., glued, adhered, bonded, etc.) to form the shunting element 102. More specifically, a lower surface of the first layer 110 is sealed to an upper surface of the second layer 112, and a lower surface of the second layer 112 is sealed to an upper surface of the third layer 114. Sealing the layers prevents or at least reduces fluid from leaking through the system 100 between layers. Additional details regarding multi-layered shunting systems are described in International Patent Application No. PCT/US2022/037917, the disclosure of which is incorporated by reference herein in its entirety.

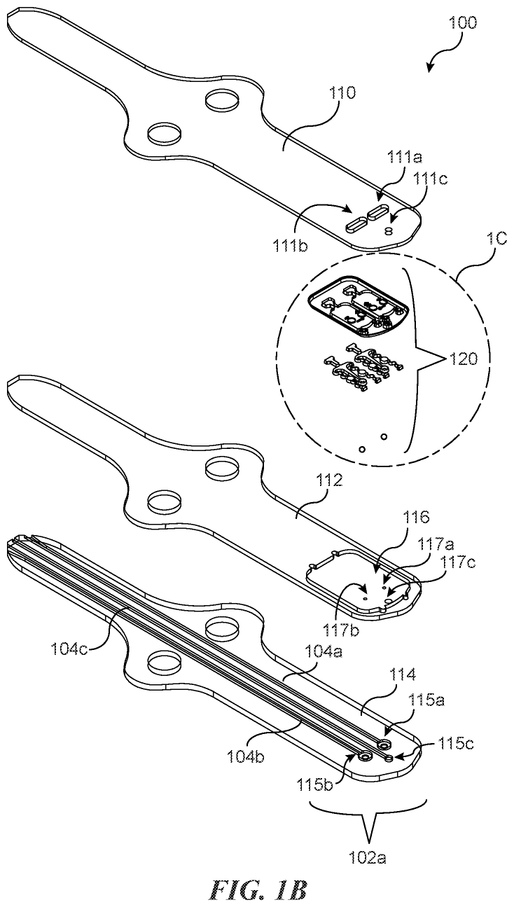

The first layer 110 includes several openings (e.g., windows, ports, apertures, etc.). More specifically, the first layer 110 includes a first opening 111a, a second opening 111b, and a third opening 111c (collectively referred to as the openings 111). The openings 111 can have the same or different shapes and/or sizes. For example, in the illustrated embodiment, the first opening 111a and the second opening 111b have a generally similar shape and size, while the third opening 111c has a different shape (e.g., round vs. oval) and size (e.g., smaller). In operation, the openings 111 permit fluid to flow into the system 100. More specifically, and as described in greater detail below, the first opening 111a permits fluid to flow into the first channel 104a, the second opening 111b permits fluid to flow into the second channel 104b, and the third opening 111c permits fluid to flow into the third channel 104c. In addition to permitting fluid to flow into the system 100, the openings 111 can enable a user to view and/or actuate the actuation assembly 120. For example, when the system 100 is in an assembled configuration, the first opening 111a can be at least partially aligned with a first actuator 124a of the actuation assembly 120, and the second opening 111b can be at least partially aligned with a second actuator 124b of the actuation assembly 120. As described in greater detail with reference to FIG. 2A, during operation of the system 100, a user can actuate the first actuator 124a or the second actuator 124b by directing energy (e.g., laser energy) through the first opening 111a or the second opening 111b, respectively.

The second layer 112 includes a chamber or cavity 116 at the first end portion 102a, with an opening to the chamber 116 facing toward the first layer 110. The chamber 116 provides an empty space or cavity for receiving the actuation assembly 120. The chamber 116 also includes several openings (e.g., ports, apertures, etc.) that generally align with the openings 111 of the first layer 110. For example, the chamber 116 includes a first aperture 117a, a second aperture 117b, and a third aperture 117c (collectively referred to as the apertures 117). The apertures 117 extend fully through the second layer 112 such that fluid can flow through the second layer 112 via the apertures 117. Similar to the openings 111, the apertures 117 can have the same or different shapes and/or sizes. In the illustrated embodiment, the first aperture 117a and the second aperture 117b have generally the same shape and size, while the third aperture 117c has generally the same shape (e.g., round) but a larger size (e.g., diameter). The first aperture 117a is fluidly connected to both the first opening 111a and the first channel 104a such that fluid flowing into the system 100 via the first opening 111a can flow into the first channel 104a via the first aperture 117a. Similarly, the second aperture 117b is fluidly connected to both the second opening 111b and the second channel 104b, such that fluid flowing into the system 100 via the second opening 111b can flow into the second channel 104b via the second aperture 117b. The third aperture 117c is fluidly connected to both the third opening 111c and the third channel 104c such that fluid flowing into the system 100 via the third opening 111c can flow into the third channel 104c via the third aperture 117c.

The third layer 114 defines or at least partially defines the channels 104. For example, the void space of the channels 104 can be formed within the third layer 114, although the second layer 112 can form a “top” of the channels 104 (e.g., the channels become closed off once the second layer 112 is sealed to the third layer 114). The third layer 114 also defines a first well 115a fluidly coupled to the first channel 104a at the first end portion 102a, a second well 115b fluidly coupled to the second channel 104b at the first end portion 102a, and a third well 115c fluidly coupled to the third channel 104c at the first end portion 102a. The first well 115a is aligned with, and therefore configured to receive fluid from, the first aperture 117a of the second layer 112. The second well 115b is aligned with, and therefore configured to receive fluid from, the second aperture 117b of the second layer 112. The third well 115c is aligned with, and therefore configured to receive fluid from, the third aperture 117c. In the illustrated embodiment, each of the wells 115 has a circular cross-sectional shape. In other embodiments, however, one or more of the wells 115 can have a different shape. For example, in some embodiments the first well 115a and/or the second well 115b has an oval shape and/or an elongated channel-like shape. In such embodiments, the elongated portion of the well 115 can extend generally normal to an axial length of the system 100, and may be at least partially curved.

As described above and as best shown in FIG. 1C, the actuation assembly 120 includes a first actuator 124a and a second actuator 124b (collectively referred to as the actuators 124). Referring to FIGS. 1B and 1C together, the first actuator 124a can be configured to selectively control the fluid resistance and/or flow of fluid through the first aperture 117a of the second layer 112 (and thus through the first channel 104a), and the second actuator 124b can be configured to selectively control the fluid resistance and/or the flow of fluid through the second aperture 117b of the second layer 112 (and thus through the second channel 104b). More specifically, the first actuator 124a can be selectively moveable between (a) a first (e.g., open) position or configuration in which the first actuator 124a does not block or at least does not substantially block, and therefore permits fluid flow through, the first aperture 117a, and (b) a second (e.g., closed or at least partially closed) position or configuration in which the first actuator 124a substantially blocks and/or seals, and therefore does not permit flow or at least clinically meaningful flow, through the first aperture 117a. That is, the first actuator 124a imparts a greater fluidic resistance through the first aperture 117a when the first actuator 124a is in the second position relative to when the first actuator 124b is in the first position. Likewise, the second actuator 124b can be selectively moveable between (a) a first (e.g., open) position in which the second actuator 124b does not block or at least does not substantially block, and therefore permits fluid flow through, the second aperture 117b, and (b) a second (e.g., closed or at least partially closed) position in which the second actuator 124b substantially blocks and/or seals, and therefore does not permit flow or at least clinically meaningful flow, through the second aperture 117b.

As also best shown in FIG. 1C, the actuation assembly 120 can also include a plate, cartridge, or backbone 122 configured to hold and prime the actuators 124 and positionable within the chamber 116 of the second layer 112. For example, the plate 122 can include a first actuator chamber 123a configured to receive the first actuator 124a and a second actuator chamber 123b configured to receive the second actuator 124b (collectively referred to as the actuator chambers 123; the openings to the actuator chambers 123 are facing downwardly toward, and thus configured to receive, the actuators 124 in the orientation shown in FIG. 1C). The actuator chambers 123 can be sized and shaped such that they at least partially deform (e.g., stretch, tension, compress, etc.) the actuators 124 when the actuators 124 are positioned therein. In embodiments in which the actuators 124 are composed of a shape memory material, this deformation primes the actuators 124 and permits them to be subsequently actuated, as described in greater detail with reference to FIG. 1D.

Referring again to FIGS. 1B and 1C together, the plate 122 also includes one or more first plate openings 121a that generally align with the first opening 111a in the first layer 110, one or more second plate openings 121b that generally align with the second opening 111b in the first layer 110, and one or more third openings 121c that generally align with the third opening 111c in the first layer 110. As described below, the first plate openings 121a and the second plate openings 121b permit a user to actuate the corresponding actuators 124 (e.g., by providing a line-of-sight to a portion of the corresponding actuator 124), while each of the openings 121 permit fluid to flow through the system 100, as described below.

For example, in addition to housing the actuators 124, the actuator chambers 123 also form part of the fluid flow path through the system 100. For example, the first actuator chamber 123a is (a) fluidly coupled to the first opening 111a in the first layer 110 via the first plate opening(s) 121a, and (b) fluidly coupled to the first aperture 117a in the second layer 112, such that fluid can flow between the first opening 111a and the first aperture 117a via the first plate opening(s) 121a and the first actuator chamber 123a. Likewise, the second actuator chamber 123b is (a) fluidly coupled to the second opening 111b in the first layer 110 via the second plate opening(s) 121b, and (b) fluidly coupled to the second aperture 117b in the second layer 112, such that fluid can flow between the second opening 111b and the second aperture 117b via the second plate opening(s) 121b and second actuator chamber 123b. In some embodiments, the actuator chambers 123 are fluidly isolated. In other embodiments, the actuator chambers 123 are fluidly connected.

The plate 122 can be composed of a material that has generally stiffer mechanical properties than the layers 110, 112, 114, and/or the actuators 124. For example, the plate 122 can be composed of superelastic Nitinol, stainless steel, titanium, glass, plastic, or other suitable materials. This is expected to enable the plate 122 to resist deformation when the actuators 124 are deformed and coupled to the plate 122, as described in greater detail below. This feature is also expected to enable the plate 122 to resist upward deflection of the actuators 124, which can assist in improving fluid flow control through the system 100.

Each actuator 124 also can include a sealing element 130, which is shown separately from the actuators 124 in the exploded view of FIG. 1C for ease of illustration. More specifically, the first actuator 124a includes a first sealing element 130a and the second actuator 124b includes a second sealing element 130b. The sealing elements 130 can be composed of a generally noncompressible material such as glass, plastic, stainless steel, or the like. In other embodiments, the sealing elements 130 can be composed of a partially elastic material, such as silicone, rubber, or the like. Without intending to be bound by theory, the sealing elements 130 are expected to improve the fluid blocking effect (e.g., seal) of the actuators 124 at the corresponding first aperture 117a and second aperture 117b when the actuators 124 are in the closed position. Additional details regarding sealing elements and mechanisms that can be used with the systems 100 are described in U.S. Provisional Patent Application Nos. 63/338,393 and 63/421,851, the disclosures of which are incorporated by reference herein in their entireties.

FIG. 1D is an enlarged view of the first actuator 124a and the first sealing element 130a, with other aspects of the system 100 omitted for purposes of illustration. The first actuator 124a includes a projection or gating element 132 having a distal end portion 132a configured to at least partially control (e.g., gate) flow through the system 100. To do so, the distal end portion 132a includes a sealing element retention feature 135 configured to hold and retain the first sealing element 130a (the first sealing element 130a is shown removed from the sealing element retention feature in FIG. 1D for purposes of illustration).

The first actuator 124a further includes a first actuation element 138a and a second actuation element 138b. The first actuation element 138a can be configured to rotate, pivot, slide, or otherwise move the gating element 132, and thus the first sealing element 130a, in a first direction. For example, when actuated, the first actuation element 138a can be configured to move the gating element 132 from the first (e.g., open) position to and/or toward the second (e.g., closed) position. The second actuation element 138b can be configured to selectively rotate, pivot, slide, or otherwise move the gating element 132, and thus the first sealing element 130a, in a second direction generally opposite the first direction. For example, when actuated, the second actuation element 138b can be configured to move the gating element 132 form the second (e.g., closed) position to and/or toward the first (e.g., open) position.

In some embodiments, the first actuation element 138a and the second actuation element 138b can be composed at least partially of a shape memory material or alloy (e.g., Nitinol). Accordingly, the first actuation element 138a and the second actuation element 138b can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element 138a and the second actuation element 138b may have reduced (e.g., relatively less stiff) mechanical properties that cause the actuation elements to be more easily deformable (e.g., compressible, expandable, etc.) relative to when the actuation elements are in the first material state. In the second material state, the first actuation element 138a and the second actuation element 138b may have increased (e.g., relatively more stiff) mechanical properties relative to the first material state, causing an increased preference toward a specific preferred geometry (e.g., original geometry, manufactured or fabricated geometry, heat set geometry, etc.).

The first actuation element 138a and the second actuation element 138b can be selectively and independently transitioned between the first material state and the second material state by applying energy (e.g., laser energy, electrical energy, etc. delivered from an energy source external to the system 100 and a patient in which the system 100 is implanted) to the first actuation element 138a or the second actuation element 138b to heat the corresponding actuation element above a transition temperature (e.g., above an austenite finish (Af) temperature, which is generally greater than body temperature). If the first actuation element 138a (or the second actuation element 138b) is deformed relative to its preferred geometry when heated above the transition temperature, the first actuation element 138a (or the second actuation element 138b) will move to and/or toward its preferred geometry. In some embodiments, the first actuation element 138a and the second actuation element 138b are operably coupled such that, when the actuated actuation element (e.g., the first actuation element 138a) transitions toward its preferred geometry, the non-actuated actuation element (e.g., the second actuation element 138b) is further deformed relative to its preferred geometry. Additional details regarding, and examples of, bi-directional shape memory actuators that can be used with the present technology are described in U.S. Patent Application Publication Nos. 2020/0229982 and 2021/0251806 and International Patent Application No. PCT/US23/71106, the disclosures of which are incorporated by reference herein in their entireties and for all purposes.

The first actuator 124a further includes a first anchoring element 140, a second anchoring element 141, and a third anchoring element 142 (collectively referred to as the anchoring elements 140-142). To couple the first actuator 124a to the system 100, the anchoring elements 140-142 can be secured to (e.g., placed within) corresponding anchoring features in the first actuator chamber 123a of the actuation assembly 120 (FIG. 1C). In some embodiments, the first actuation element 138a and the second actuation element 138b are deformed relative to their preferred geometries (e.g., “loaded”) when the first actuator 124a is positioned within the first actuator chamber 123a. For example, the first actuator chamber 123a can be configured/dimensioned such that the act of placing the anchoring elements 140-142 within the corresponding anchoring features deforms the first actuation element 138a and the second actuation element 138b relative to their original manufactured geometries. In some embodiments positioning the anchoring elements 140-142 within corresponding anchoring features can increase a length of the actuation elements 138 (e.g., tension) relative to their preferred geometries. In other embodiments, positioning the anchoring elements 140-142 within corresponding anchoring features can decrease a length of the actuation elements 138 (e.g., compress) relative to their preferred geometries. Additional details regarding loading and deforming shape memory actuators are described in U.S. Patent Application Publication No. 2021/0251806, previously incorporated by reference herein, and International Patent Application No. PCT/US21/49140, the disclosure of which is incorporated by reference in its entirety and for all purposes.

As described above, the distal end portion 132a of the gating element 132 is configured to moveably interface with various features of the system 100 to at least partially control the flow of fluid through one or more flow pathways extending through the system 100. For example, referring collectively to FIGS. 1B-1D, when the first actuator 124a is positioned within the first actuator chamber 123a, the distal end portion 132a of the gating element 132 is positioned proximate the first aperture 117a in the second layer 112 of the shunting element 102. As a result, the first actuator 124a can selectively move the sealing assembly 233 between the first (e.g., open) position in which the sealing assembly 233 does not block or substantially block flow through the first aperture 117a, and the second (e.g., closed) position in which the sealing assembly 233 blocks, or at least partially blocks, fluid flow through the first aperture 117a. In this way, the first actuator 124a can control the flow of fluid through the first channel 104a.

Flow through the second channel 104b can be controlled in the same or generally similar manner as flow through the first channel 104a. For example, the second actuator 124b can be the same as or generally similar to the first actuator 124a, but can be positioned within the second actuator chamber 123b such that the second actuator 124b is proximate the second aperture 117b in the second layer 112 of the shunting element 102. In contrast to the first channel 104a and the second channel 104b, the third channel 104c is designed to be “always open” such that it permits at least some degree of fluid flow through the system 100 even when both the first channel 104a and the second channel 104b are blocked/closed. Of course, the present technology is not limited to particular combinations of “always open” and adjustable channels, and can include more or fewer of each channel type. Similarly, although described as having two actuators 124, the system 100 can have more or fewer actuators, such as one, three, four, or more.

### B. Select Embodiments of System State Indicators

As described above, the present technology includes shunting systems with actuators that can be selectively actuated to adjust a level of therapy provided by the shunt. In some embodiments, a physician or other healthcare provider can adjust the shunt after the shunt is implanted in the patient (e.g., in vivo adjustments). That is, the physician or other healthcare provider can monitor the patient over a period (e.g., days, weeks, months, years, etc.) and periodically adjust the shunt based on a change in a patient condition. For example, in the context of treating glaucoma, a physician may monitor an intraocular pressure in the patient's eye. If the intraocular pressure is too high, the physician can adjust the shunt to provide an increased level of therapy (e.g., by decreasing the resistance through the shunt to increase fluid drainage via the shunt). If the intraocular pressure is too low, the physician can adjust the shunt to provide a decreased level of therapy (e.g., by increasing resistance through the shunt to decrease fluid drainage via the shunt).

However, depending on the size of the shunting system, the configuration of the shunting system, and/or the implant location of the shunting system, it may be difficult for the physician or other healthcare provider to determine a state of the shunting system (e.g., whether the shunt is set to an “open” or “closed” position, etc.) simply by viewing the system. For example, for the system 100 described with reference to FIGS. 1A-1D, the plate 122 sits “above” the actuators 124. Accordingly, in embodiments in which the plate 122 is not transparent (e.g., if the plate 122 is composed of Nitinol or another non-transparent material), the plate 122 may partially or fully block a user from directly seeing the actuators 124 to determine whether the actuators 124 are in the first (e.g., open) position or the second (e.g., closed) position. Relatedly, if a user cannot easily see the actuators 124, a user may not know which portion of the actuator 124 to actuate to induce a desired change in a state of the system 100.

The present technology is expected to address one or more of the foregoing issues associated with determining a state of an adjustable shunting system. In particular, adjustable shunting systems configured in accordance with the present technology can include visual state indicators that enable a physician or other healthcare provider to quickly and easily (a) determine a current state of the shunt, (b) determine which actuator and/or actuation element to actuate to provide a desired change in therapy, and (c) confirm that the intended adjustment occurred following actuation.

FIG. 2A, for example, is an enlarged view of the plate 122 of the system 100 described with reference to FIGS. 1A-1D. The plate 122 includes system state indicators 256 (“state indicators 256”) to assist a user in determining a current position or state of the first actuator 124a and the second actuator 124b (FIGS. 1A-1D). As described above with reference to FIG. 1C, the first and second actuators 124a and 124b are positioned in the first actuator chamber 123a and the second actuator chamber 123b, respectively, when the system 100 is assembled to control the flow of fluid through the first aperture 117a and the first channel 104a (FIG. 1B). The plate 122 includes a first actuator position indicator 256a for determining a position of the first actuator 124a (FIGS. 1B and 1C), and thus a state (e.g., open or closed) of the first channel 104a. The first actuator position indicator 256a includes a plurality of holes 257 extending through the plate 122 and thus providing a line-of-sight through the plate 122. This enables a user to see “beneath” the plate 122 even in embodiments in which the plate 122 is not transparent. The holes 257 include a first relatively larger hole 257a and one or more second relatively smaller holes 257b.

Depending on the position of the first actuator 124a, the gating element 132 can be seen through one or more of the holes 257. For example, FIGS. 2B and 2C are enlarged, cut-away views of the first actuator position indicator 256a and a portion of the gating element 132. As shown in FIG. 2B, the gating element 132 (the outline of which is shown in broken line) is positioned beneath, and thus can be seen through, the first relatively larger hole 257a when the gating element 132 is in the second (e.g., closed) position. In contrast, and as shown in FIG. 2C, the gating element 132 (the outline of which is shown in broken line) is positioned beneath, and thus can be seen through, the second relatively smaller holes 257b when the gating element 132 is in the first (e.g., open) position. Accordingly, if the gating element 132 can be seen through the first relatively larger hole 257a as shown in FIG. 2B, a user knows that the first actuator 124a is in the second (e.g., closed) position and, as a result, there is little or no flow occurring through the first channel 104a. Conversely, if the gating element 132 can be seen through the second relatively smaller holes 257b as shown in FIG. 2C, a user knows that the first actuator 124a is in the first (e.g., open) position and, as a result, the first channel 104a is open for fluid flow. This enables a user to quickly and easily determine a position of the first actuator 124a, and thus a state (e.g., open to flow or closed to flow) of the first channel 104a, simply by examining the first actuator position indicator 256a. Although shown as having a plurality of holes 257, in other embodiments the first actuator position indicator 256a can have other suitable configurations for conveying a position of the first actuator 124a and thus a state of the first channel 104a. For example, the first actuator position indicator 256a could be a transparent window formed in the plate 122, a single slot through the plate 122 that extends laterally, or the like.

Referring again to FIG. 2A, the plate 122 also includes a second actuator position indicator 256b for determining a position of the second actuator 124b (FIGS. 1B and 1C), and thus a state (e.g., open or closed) of the second channel 104b. The second actuator position indicator 256b can be similar to or the same as the first actuator position indicator 256a and enable a user to quickly and easily determine if the second actuator 124b is in the first (e.g., open) position or the second (e.g., closed) position.

As set forth above with reference to FIG. 1D, the first actuator 124a also includes two actuation elements: a first actuation element 138a that, when actuated, moves the gating element 132 toward the second (e.g., closed) position, and a second actuation element 138b that, when actuated moves the gating element 132 toward the first (e.g., open) position. To assist in determining which actuation element to actuate to induce a desired adjustment, the plate 122 also includes actuation or adjustment indicators that instruct where to actuate (e.g., where to direct energy, such as laser energy) to make a desired adjustment. In particular, the plate 122 includes a first decrease flow/increase resistance indicator 252a and a first increase flow/decrease resistance indicator 254a associated with the first actuator 124a (the indicators 252a and 254a can also be referred to generally as “adjustment indicators”). The first decrease flow indicator 252a is positioned directly adjacent to a first opening 121a₁ of the first plate openings 121a, and the first increase flow indicator 254a is positioned directly adjacent to a second opening 121a₂ of the first plate openings 121a. In the illustrated embodiment, the first decrease flow indicator 252a is shaped as a minus or dash, although other suitable shapes can be used. The first increase flow indicator 254a is shaped as a double chevron, although other suitable shapes (e.g., single chevron, plus sign, circle, etc.) can be used. In some embodiments, the first decrease flow indicator 252a and the first increase flow indicator 254a can be colored to visibly stand out from the plate 122. For example, the first decrease flow indicator 252a and the first increase flow indicator 254a can have a different color than either or both of the plate 122 or the shunting element 102.

The first decrease flow indicator 252a indicates that, to decrease flow through the first channel 104a (FIGS. 1A and 1B), energy (e.g., laser energy) should be directed through the first opening 121a₁. Because the first opening 121a₁ aligns with the first actuation element 138a of the first actuator 124a (FIGS. 1C and 1D), directing energy through the first opening 121a₁ heats/activates the first actuation element 138a. That is, directing energy through the first opening 121a₁ can heat the first actuation element 138a above its transition temperature. If the gating element 132 (FIG. 1D) is in the first (e.g., open) position when the first actuation element 138a is heated above its transition temperature, the first actuation element 138a will change in shape (e.g., decrease in length if under tension) and cause the gating element 132 to rotate toward the second (e.g., closed) position, as described above with reference to FIGS. 1C and 1D. This increases the fluid resistance through the first aperture 117a (FIG. 1B), and therefore decreases flow through the first channel 104a. Accordingly, the first decrease flow indicator 152a can assist a user in identifying where to direct energy to decrease flow through the first channel 104a of the system 100.

The first increase flow indicator 254a indicates that, to increase flow through the first channel 104a, energy should be directed through the second opening 121a₂. Because the second opening 121a₂ aligns with the second actuation element 138b of the first actuator 124a, directing energy through the second opening 121a₂ heats/activates the second actuation element 138b. That is, directing energy through the second opening 121a₂ can heat the second actuation element 138b above its transition temperature. If the gating element 132 is in the second (e.g., closed) position when the second actuation element 138b is heated above its transition temperature, the second actuation element 138b will change in shape (e.g., decrease in length if under tension) and cause the gating element 132 to rotate toward the first (e.g., open) position, as described above. This decreases fluid resistance through the first aperture 117a and therefore increases flow through the first channel 104a. Accordingly, the first increase flow indicator 154a can assist a user in identifying where to direct energy to increase flow through the first channel 104a.

The plate 122 further includes a second decrease flow/increase resistance indicator 252b and a second increase flow/decrease resistance indicator 254b that are associated with the second actuator 124b. The second decrease flow indicator 252b can have a similar function as the first decrease flow indicator 252a, except that the second decrease flow indicator 252b is associated with the second actuator 124b instead of the first actuator 124a, and thus is associated with flow through the second channel 104b instead of the first channel 104a. Accordingly, the second decrease flow indicator 252b is positioned directly adjacent a first opening 121b₁ of the second plate openings 121b and indicates to a user that, to decrease flow through the second channel 104b, energy should be directed through the first opening 121b₁. Similarly, the second increase flow indicator 254b can have a similar function as the first increase flow indicator 254a, except that the second increase flow indicator 254b is associated with the second actuator 124b instead of the first actuator 124a, and thus is associated with flow through the second channel 104b instead of the first channel 104a. Accordingly, the second increase flow indicator 254b is positioned directly adjacent a second opening 121b₂ of the second plate openings 121b and indicates to a user that, to increase flow through the second channel 104b, energy should be directed through the second opening 121b₂.

In some embodiments, the first increase flow indicator 254a associated with the first actuation 124a and the second increase flow indicator 254b associated with the second actuator 124b can further indicate the relative level of therapy (e.g., flow) that can be provided by opening the first channel 104a and the second channel 104b, respectively. For example, in the illustrated embodiment, the first increase flow indicator 254a includes a double chevron and the second increase flow indicator 254b includes a single chevron. The double chevron indicates to the user that opening the first channel 104a (i.e., by setting the first actuator 124a to the first (e.g., open) position) provides relatively greater flow than opening the second channel 104b. For example, the first channel 104a may have a lower fluid resistance than the second channel 104b and thus provide greater fluid drainage when open.

The decrease flow indicators 252 and the increase flow indicators 254 assist a user in determining where to actuate to induce a desired adjustment, but do not provide confirmation that an intended adjustment occurred. Rather, the first actuator position indicator 256a and the second actuator position indicator 256b are also expected to enable a user to confirm that an intended adjustment to the system 100 took place. For example, if the first actuator 124a is set to a first (e.g., open) position such that fluid can flow through the first channel 104a, the user should be able to visualize the gating element 132 only through the second, relatively smaller, holes 257b of the first actuator position indicator 256a (e.g., as shown in FIG. 2C). If the user desires to reduce (e.g., stop) flow through the first channel 104a, the user knows to direct energy through the first opening 121a₁ by virtue of the first decrease flow indicator 252a, described previously. In response to the user directing energy through the first opening, the gating element 132 should transition from the first (e.g., open) position to and/or toward the second (e.g., closed) position, and thus be visible through the first, relatively larger hole 257a instead of the second, relatively smaller holes 257b (e.g., as shown in FIG. 2B). Accordingly, visual identification of the gating element 132 through the first, relatively larger, hole 257a after actuation confirms that the desired adjustment occurred. If the user does not see the gating element 132 through the first, relatively larger hole 257a after actuation, the user knows to continue directing energy through the first opening 121a₁ until the user sees the gating element 132.

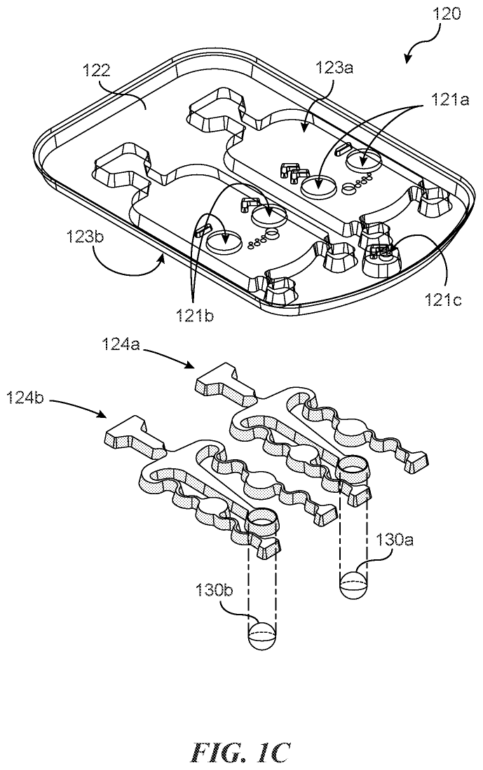

The system state indicators of the present technology can have other forms than those described with reference to the system 100 and FIGS. 2A-2C. For example, FIGS. 3A and 3B illustrate another adjustable shunting system 300 (“the system 300”) configured in accordance with select embodiments of the present technology that includes another variation of system state indicators. More specifically, FIG. 3A is a top view of the system 300 and FIG. 3B is an enlarged top view of a portion of the system 300 taken along the lines indicated in FIG. 3A. Similar to the system 100 of FIGS. 1A-2C, the system 300 is configured to provide a titratable therapy for shunting fluid from a first body region to a second body region, such as shunting aqueous from an anterior chamber of a patient's eye to a target outflow location. As described below, the system 300 also includes system state indicators that enable a user to determine a current state of the system 300, determine how to adjust the fluid resistance or therapy level provided by the system 300, and confirm that the intended adjustment to fluid resistance or therapy level occurred.

Certain features of the system 300 can be generally similar to or the same as the corresponding features of the system 100. One skilled in the art will appreciate that the description of various components of the system 100 can apply equally to like components of the system 300, unless the context clearly dictates otherwise. Referring first to FIG. 3A, the system 300 can include a shunting element 302 (e.g., an elongated housing) with a plurality of flow channels 304 (shown as a first flow channel 304a, a second flow channel 304b, and a third flow channel 304c, which are collectively referred to herein as the flow channels 304) extending therethrough. In some embodiments, the shunting element 302 is the same as, or at least similar to, the shunting element 102 of FIGS. 1A and 1B, and therefore can be composed of the same materials and/or have the same or similar components as described above with reference to FIGS. 1A and 1B. The system 300 further includes an actuation assembly 320 positioned within the shunting element 302 and configured to selectively control the flow of fluid through the channels 304. Similar to the actuation assembly 120 of the system 100 described above with reference to FIG. 1C, the actuation assembly 320 can include a plate or cartridge 322, a first shape memory actuator 324a, and a second shape memory actuator 324b (the first actuator 324a and the second actuator 324b are partially obstructed from view in FIG. 3A, but can be generally similar to or the same as the first actuator 124a and the second actuator 124b described above with reference to FIG. 1C).

Referring next to FIG. 3B, the plate 322 includes a first plate opening 321a that aligns with, and therefore provides a visual window to, at least a portion of the first actuator 324a, and a second plate opening 321b that aligns with, and therefore provides a visual window to, at least a portion of the second actuator 324b (collectively referred to as “the plate openings 321”). During operation, a user can actuate the actuators 324 by directing laser energy through the plate openings 321 to heat specific regions of the actuators 324 above their transition temperature, which can selectively adjust a position of the actuators 324. As described above with reference to the system 100 and FIGS. 1A-1C, adjusting a position of the actuators 324 changes a fluid resistance of the system 300 (e.g., by causing a gating element 332 of the actuators 324 to move relative to an inflow or outflow port.

The plate 322 also includes system state indicators 356 (“the state indicators 356”) to assist a user in determining a current position or state of the first actuator 324a and the second actuator 324b. Unlike the state indicators 156 of the system 100, the state indicators 356 (shown as a first state indicator 356a and a second state indicator 356b) each include a single opening or window 357 (shown as a first opening 357a and a second opening 357b) with a marker 358 (shown as a first marker 358a and a second marker 358b). The markers 358 can include a tab, projection, notch, groove, or other visual marking or cue. For example, although shown as a tooth extending in the same plane as, and thus at least partially defining a perimeter of, the openings 357, in other embodiments the markers 358 can be etched, drawn, or otherwise deposited upon a portion of the plate 322 adjacent the openings 357.

The markers 358 aid a user (e.g., physician) in determining a position of the corresponding gating element 332 relative to the aperture (not shown) that it gates. For example, when the gating element 332 of the actuators 324 is in the second (e.g., closed) position, the gating element 332 aligns with the corresponding marker 358. When the gating element 332 of the actuators 324 is in the first (e.g., open) position, the gating element 332 does not align with the marker (as shown in FIG. 3B). Thus, a user (e.g., physician) can quickly and easily determine if each actuator is in the “open” or “closed” position by examining whether the gating element 332 aligns with the marker 358. Although the foregoing describes alignment between the marker 358 and the gating element 332 as indicating the “closed” position, in other embodiments alignment between the marker 358 and the gating element 332 can indicate the “open” position. In yet other embodiments, each state indicator 356 can each include two markers 358, with a first marker designating the “open” position and a second marker designating the “closed” position.

The plate 322 also includes actuation or adjustment indicators similar to the plate 122 of the system 100 (FIG. 2A) that instruct a user where to actuate to change fluid resistance through the system 300. In particular, the plate 322 includes a first increase flow/decrease resistance indicator 354a associated with the first actuator 324a, and a second increase flow/decrease resistance indicator 354b associated with the second actuator 324b (collectively referred to as “the increase flow indicators 354”). As described in detail above with reference to FIG. 2A, the increase flow indicators 354 indicate which portion of the actuators 324 energy should be applied to in order to increase the flow and decrease the resistance associated with the particular actuator. For example, the first increase flow indicator 354a indicates which portion (e.g., which actuation element) of the first actuator 324a should be targeted to increase the flow through the first channel 304a (FIG. 3A). Similarly, the second increase flow indicator 354b indicates which portion (e.g., which actuation element) of the second actuator 324b should be targeted to increase the flow through the second channel 304b. The increase flow indicators 254 also indicate the relative level of therapy (e.g., flow and/or resistance) that can be provided by utilizing a single chevron for the first increase flow indicator 354a and a double chevron for the second increase flow indicator 354b (e.g., the double chevron indicates opening the second actuator 324b increases flow/decreases resistance through the shunt more than opening the first actuator 324a). Unlike the plate 122 of the system 100, the plate 322 of the system 300 does not include “decrease flow/increase resistance” indicators. Rather, the absence of any indicator instructs a user which portion of the actuators 324 to target to decrease flow/increase resistance through the corresponding flow channel. Following actuation, a user can confirm an intended adjustment occurred by examining the state indicators 356.

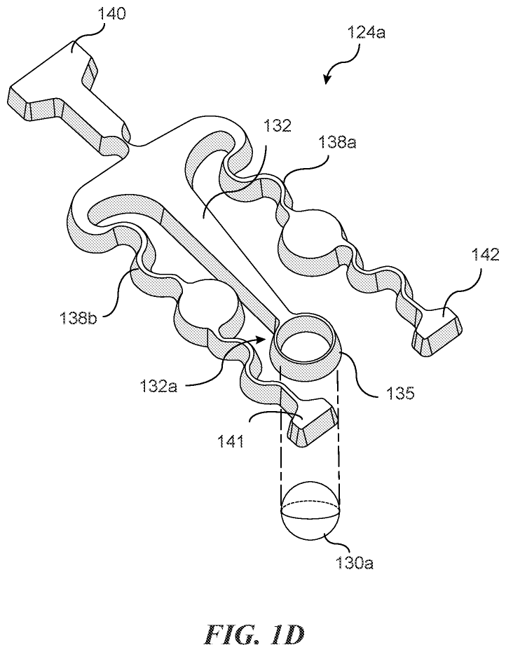

FIGS. 4A-4F illustrate additional embodiments of system state indicators configured in accordance with select embodiments of the present technology. More specifically, FIGS. 4A-4F illustrate plates 422a-f (collectively, “the plates 422”) with different system state indicators 456a-f (collectively, “the state indicators 456”) to assist a user in determining a current position or state of an actuator gating element 432. As one skilled in the art will appreciate from the foregoing description, the plates 422 and the actuator gating element 432 can be generally similar to or the same as the embodiments of these features described above with reference to FIGS. 1A-3B, and so the following description focuses on the state indicators 456.

Referring first to FIG. 4A, the state indicator 456a includes a single opening or window 457a with a marker 458a. Similar to the marker 358 of FIG. 3B, the marker 458a can include a tab, projection, notch, groove, or other visual marker or cue. Also similar to the marker 358, the marker 458a can aid a user in determining a position of the actuator gating element 432. For example, when the gating element 432 is in the second (e.g., closed) position, the gating element 432 aligns with the marker 458a, and when the gating element 432 is in the first (e.g., open) position, the gating element 432 does not align with the marker 458a. Relative to the embodiment shown in FIG. 3B, however, the opening 457a is larger in the x-direction, which may enable a user to visualize more of the gating element 432. The opening 457a can also have other suitable shapes beyond those shown in FIG. 4A, including circular, square, rectangular, or other shapes.

Referring next to FIG. 4B, the state indicator 456b also includes an opening or window 457b with a marker 458b. However, unlike the marker 458a of FIG. 4A, the marker 458b includes two bridge or extension elements that extend across the opening 457b. In the illustrated embodiment, the bridge elements form a general “X” shape, although other patterns and/or shapes are possible. When the gating element 432 is in the second (e.g., closed) position, the gating element 432 aligns with the marker 458b (e.g., the gating element 432 is positioned under the X formed by the bridge elements), indicating to a user that the gating element 432 is in the closed position. When the gating element 432 is in the first (e.g., open) position, the gating element is not aligned with the marker 458b (e.g., the gating element 432 is not positioned under the X formed by the bridge elements), indicating to the user that the gating element 432 is in the open position. In some embodiments, the bridge elements may provide the additional advantage of helping keep the gating element 432 in its desired operational plane, e.g., by reducing the likelihood that a portion of the gating element 432 inadvertently is displaced through the opening 457b.

Referring next to FIG. 4C, the state indicator 456c also includes an opening or window 457c with a marker 458c. Relative to the markers 458a and 458b of FIGS. 4A and 4B, the marker 458c exists in the same plane as the actuator gating element 432. That is, rather than being formed as part of an upper surface of the plate, the marker 458c is formed under the plate 422c (e.g., extending from a lower surface of the plate 422c or from another portion of the adjustable shunting system, not shown). The marker 458c can be a tab, projection, or other visual marker or cue, and can function similarly to the markers 458a and 458b of FIGS. 4A and 4B. For example, when the gating element 432 is in the second (e.g., closed) position, the gating element 432 aligns with the maker 458c, and when the gating element 432 is in the first (e.g., open) position, the gating element 432 does not align with the marker 458c.

The state indicator 456d shown in FIG. 4D includes an opening or window 457d having a first opening portion 457d1 and a second opening portion 457d2. The first opening portion 457d1 is shown as having an oval or pill-shape and the second opening portion 457d2 is shown as having a generally triangular shape, although other shapes are possible. Rather than having a discrete marker as in the embodiments described with reference to FIGS. 4A-4C, the first opening portion 457d1 and the second opening portion 457d2 act as the markers. For example, when the gating element 432 is in the second (e.g., closed) position, the gating element aligns with, and is therefore visual through, the first opening 457d1. When the gating element 432 is in the first (e.g., open) position, the gating element aligns with, and is therefore visual through, the second opening 457d2. Thus, a user can quickly determine whether the gating element is in the “open” or “closed” state by seeing which opening 457d the gating element is visible through.

The state indicator 456e shown in FIG. 4E also includes an opening or window 457e having a first opening portion 457e1 and a second opening portion 457e2. Relative to the embodiment shown in FIG. 4D, however, the first opening portion 457e1 and the second opening portion 457e2 are not connected (e.g., are discontinuous or discrete openings). The state indicator 456a can otherwise function similarly to the state indicator 456d of FIG. 4D, with a user being able to determine whether the gating element 432 is in the “open” or “closed” state based on which opening 457e the gating element 432 can be seen through. Without intending to be bound by theory, having separate opening portions 457e1 and 457e2 may reduce the likelihood that the gating element 432 is simultaneously visible through both opening portions, which in turn may reduce the likelihood a user misinterprets the state of the gating element 432.

The state indicator 456f shown in FIG. 4F is generally similar to the state indicator 456e of FIG. 4E. For example, the state indicator 456f includes a first opening portion 457f1 and a second opening portion 457f2 that is not connected to the first opening portion 457f1. Relative to the state indicator 456e of FIG. 4E, however, the first opening portion 457f1 is itself comprised of a plurality of smaller, discrete openings. Without intending to be bound by theory, dividing the first opening portion 457f1 into a plurality of smaller, discrete openings may assist a user in recalling which opening 457f corresponds to the gating element 432 being in the “closed” state.

As set forth above, any of the state indicators 456 described with reference to FIGS. 4A-4F can be incorporated into the system 100 of FIGS. 1A-1D to assist a user with determining a state of the actuator. These state indicators 456 can also be used in combination with any of the actuation or adjustment indicators described herein, such as the decrease flow indicators 252 and the increase flow indicators 254 of FIG. 2A, and/or the increase flow indicators 354 of FIG. 3B. Moreover, although only a single state indicator 456 was described for each plate 422 in FIGS. 4A-4F, the plates 422 can include additional state indicators, e.g., such that each actuator includes a corresponding state indicator 456 (e.g., in each illustrated embodiment, two state indicators 456 are shown). The additional state indicator(s) can be similar to or the same as the other corresponding state indicator that was described with reference to the particular Figure.

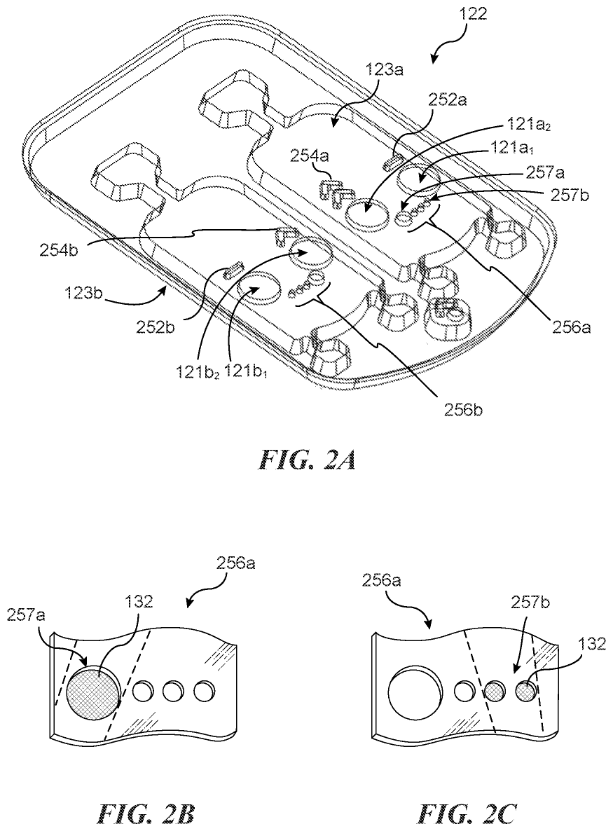

FIG. 5 is a schematic illustration of another system state indicator 556 (“the state indicator 556”) shown in isolation and configured in accordance with select embodiments of the present technology. The state indicator 556 can comprise one or more visible geometric shapes or patterns that change shape and/or size in response to an actuator (not shown) such as the actuator 124 of FIGS. 1C and 1D moving between open and closed positions. For example, in the illustrated embodiment, the state indicator 556 forms a relatively larger circle or aperture when the corresponding actuator is in the first (e.g., open) position. The state indicator 556 can automatically change a shape or size in response to the actuator being transitioned to the second (e.g., closed) position. For example, the state indicator 556 can transition to a relatively smaller circle or aperture to indicate that the actuator is in the second (e.g., closed) state. The state indicator 556 can repeatedly toggle back and forth between its relatively larger shape and relatively smaller shape in response to the actuator being toggled back and forth between the first (e.g., open) position and the second (e.g., closed) position.

Although shown as a circle, the state indicator 556 can have other shapes or sizes. In some embodiments, the state indicator 556 may fully or at least substantially fully close when the actuator is in the second (e.g., closed) position. Without intending to be bound by theory, the state indicator 556 is expected to provide an intuitive mechanism for indicating to a clinician/operator whether the actuator is in an open or closed state. As one skilled in the art will appreciate, the state indicator 556 can be incorporated into any of the adjustable shunting systems described herein, such as the systems 100 and 300 described above, or other suitable systems.

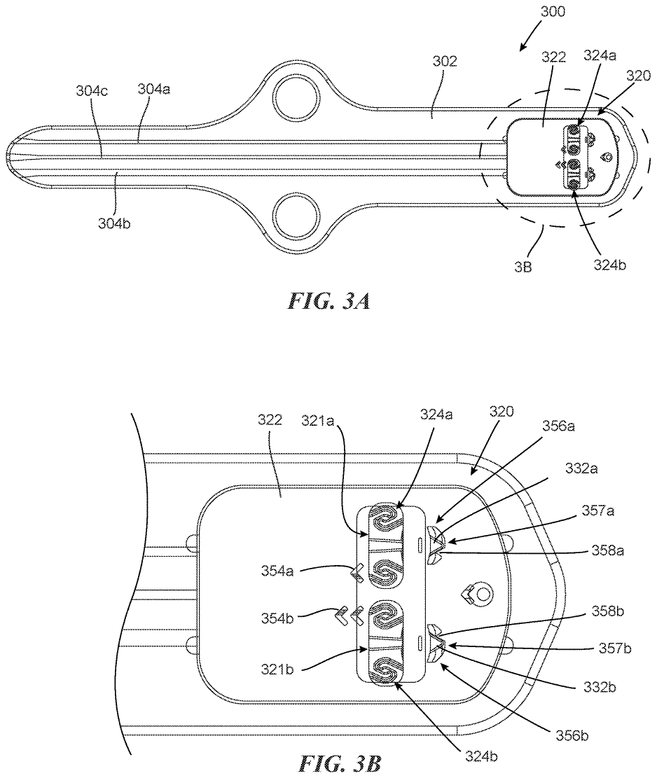

FIGS. 6A and 6B illustrate use of a system state indicator 656 (“the state indicator 656”) that is generally similar to the state indicator 556 of FIG. 5 and is also configured in accordance with select embodiments of the present technology. In particular, FIGS. 6A and 6B illustrate a portion of a gating element 632 of an actuator 624, which can be generally similar to the actuators 124 described with reference to FIGS. 1C and 1D. A distal end 632a of the gating element 632 can have a first state indicator feature 657a, which in the illustrated embodiment is a crescent or half-circle shape. Another portion of the shunting system such as a projection 622 on a plate (not shown, but can be similar to the plate 122 of FIGS. 1B and 1C) can include a second state indicator feature 657b, which can also be a crescent or half-circle shape. The first state indicator feature 657a and the second state indicator feature 657b can collectively form the state indicator 656.

FIG. 6A illustrates the actuator 624 in the first (e.g., open) position. In the first (e.g., open) position, the first state indicator feature 657a and the second state indicator feature 657b are spaced apart. As a result, the state indicator 656 appears “larger” and “open,” which indicates the actuator 624 is in the first (e.g., open) position. In contrast, FIG. 6B illustrates the actuator 624 in the second (e.g., closed) position. In the second (e.g., closed) position, the distal end 632a of the gating element 632 has moved toward the projection 622, which generally does not move because it is coupled to a fixed portion of the shunting system such as the plate 122 of FIGS. 1B and 1C. Of note, in the second (e.g., closed) position, the first state indicator feature 657a and the second state indicator feature 657b are closer together. As a result, the state indicator 656 appears “smaller” or even “closed,” which indicates the actuator 624 is in the second (e.g., closed) position.

Although primarily described in the context of the system 100 and the system 300, the present technology includes other adjustable shunts having one or more system state indicators that enable a physician or other healthcare provider to quickly and easily (a) determine a current state of the shunt, (b) determine which actuator and/or actuation element to actuate to provide a desired change in therapy, and/or (c) confirm that the intended adjustment occurred following actuation. For example, any of the system state indicators described herein can be incorporated into other adjustable shunting systems, such as those described in U.S. Patent Application Publication Nos. 2020/0229977, 2020/0229982, 2021/0251806, 2022/0142818, and 2022/0202613, each of which is incorporated by reference herein in its entirety.

### C. Select Embodiments of Flow Indicators

The present technology further includes shunting systems having one or more mechanisms for determining whether fluid is flowing through the shunt. That is, in addition to or in lieu of having one or more mechanisms for determining a state of a shunt (e.g., open to flow or closed to flow) as described above under Heading B, shunts configured in accordance with the present technology can include a flow indicator that confirms whether flow is occurring through the shunt. This is expected to be useful because it can help confirm that a blockage (e.g., via cellular or other debris) has not developed in the shunt, and that the shunt is providing therapy as intended. As one skilled in the art will appreciate from the foregoing, such flow indicators can be useful regardless of whether the shunt is adjustable. For example, flow indicators are expected to be useful in both adjustable shunts and conventional non-adjustable shunts to provide a physician or other healthcare provider with confirmation that fluid is flowing through the shunt as intended. Accordingly, any of the flow indicators described herein can be used in connection with a non-adjustable shunt, such as otherwise conventional tube shunts.

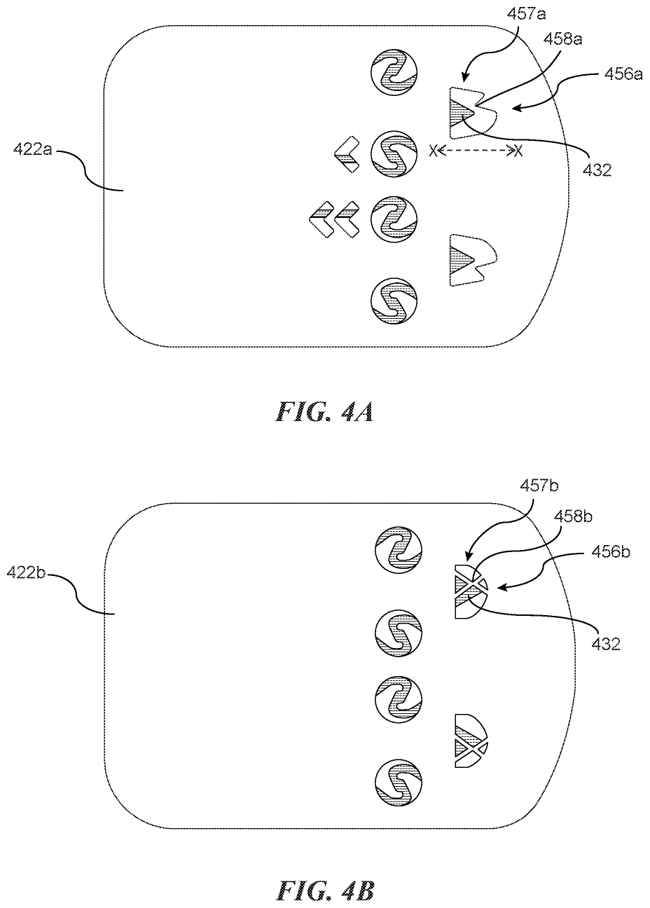

FIGS. 7-14B illustrate various different mechanisms for determining whether fluid is flowing through the shunt. Each of FIGS. 7-14B describe the flow indicators in connection with the first channel 104a of the system 100 (FIGS. 1A-1D); however, as set forth above, each of the flow indicators could be used in connection with other channels (e.g., the second channel 104b or the third channel 104c), other adjustable shunts (e.g., the adjustable shunting system 300 of FIGS. 3A and 3B), or other non-adjustable shunts.

FIG. 7 illustrates a first embodiment of a flow indicator assembly 760 for detecting fluid flow through a shunt and configured in accordance with select embodiments of the present technology. As shown, the flow indicator assembly 760 includes an eddy or offshoot 762 fluidly connected to the first channel 104a. The flow indicator assembly 760 further includes a flow indicator 770 positioned within the eddy 762. The flow indicator 770 includes a fan or blade 772 rotatably coupled to fixed portion of the system 100 via a connector 774 (e.g., a pin, a screw, etc.). In operation, some of the fluid (shown using broken-line arrows) flowing through the first channel 104a from the first well 115a can flow into the eddy 762. As fluid flows into the eddy 762, the fluid contacts the fan 772 of the flow indicator 770 and forces the flow indicator 770 to rotate. Indeed, continuous flow of fluid is expected to induce continuous or at least generally continuous rotation of the flow indicator 770. In contrast, if fluid is not flowing through the first channel 104a, fluid will not push the fan 772 and thus the flow indicator 770 will not rotate. Thus, rotation of the flow indicator 770 can be detected by a physician or other healthcare provider to confirm fluid is flowing through the first channel 104a.

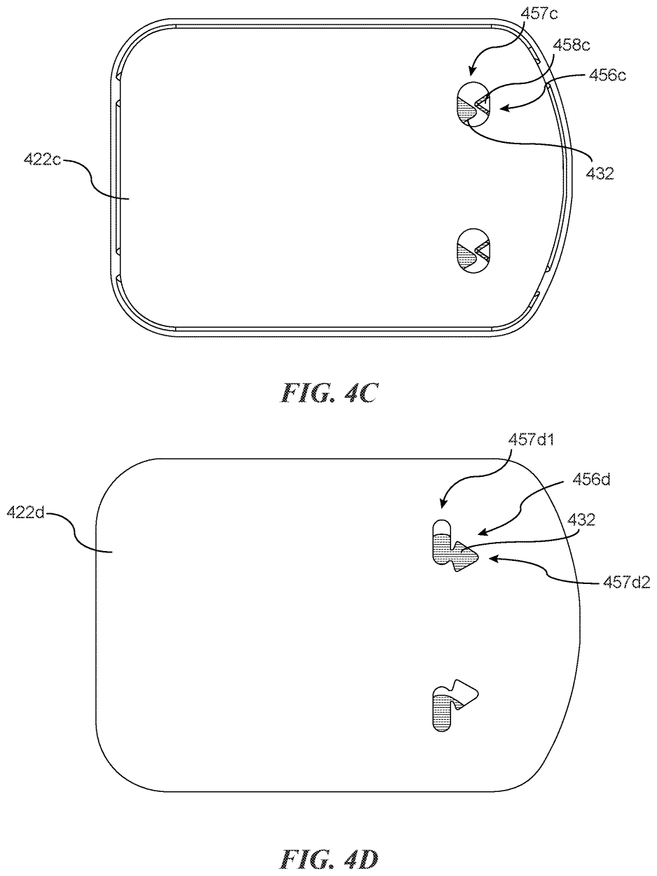

FIG. 8 illustrates a second embodiment of a flow indicator assembly 860 for detecting fluid flow through a shunt and configured in accordance with select embodiments of the present technology. As shown, the flow indicator assembly 860 includes a bypass channel portion 864 extending from and fluidly coupled to the first channel 104a. The bypass channel portion 864 includes an eddy 862, which can be generally similar to the eddy 762 described with reference to FIG. 7, except that the eddy 862 is positioned along the bypass channel portion 864 instead of being in direct fluid connection with the first channel 104a. The flow indicator assembly 860 further includes a flow indicator 870 positioned within the eddy 862. The flow indicator 870 can include a plurality of fans or blades 872 rotatably coupled to a fixed portion of the system 100 via a connector 874. In operation, some of the fluid (shown using broken-line arrows) flowing through the first channel 104a will flow into the bypass channel portion 864 and into the eddy 862. As the fluid flows into the eddy 862, the fluid contacts the blades 872 of the flow indicator 870 and forces the blades 872 to rotate. Similar to the flow indicator 770 of FIG. 7, continuous flow of fluid through the first channel 104a is expected to induce continuous or at least generally continuous rotation of the flow indicator 870. In contrast, if fluid is not flowing through the first channel 104a, fluid will not push the blades 872 and thus the flow indicator 870 will not rotate. Thus, rotation of the flow indicator 870 can be detected by a physician or other healthcare provider to confirm fluid is flowing through the first channel 104a.

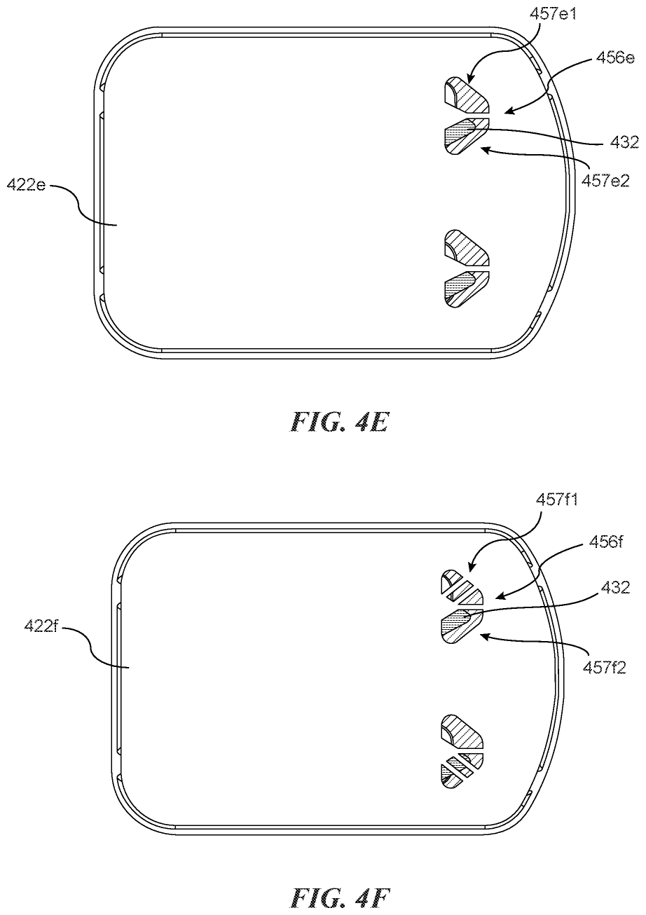

FIG. 9 illustrates a third embodiment of a flow indicator assembly 960 for detecting fluid flow through a shunt and configured in accordance with select embodiments of the present technology. Similar to the flow indicator assembly 860 of FIG. 8, the flow indicator assembly 960 includes a bypass channel portion 964 extending from and fluidly coupled to the first channel 104a. However, the flow indicator assembly 960 does not have a rotational-based flow indicator like the flow indicator assembly 860. Rather, the flow indicator assembly 960 includes a plurality of flow indicators 970 each having a flappable element 972 (e.g., a suture, string, thread, streamer, etc.) fixedly coupled to a side of the bypass channel portion 964 via a connector 674 (e.g., a needle, bar, etc.). In operation, some of the fluid (shown using broken-line arrows) flowing through the first channel 104a will flow into the bypass channel portion 964. As the fluid flows through the bypass channel portion 964, the fluid will agitate or otherwise induce motion in the flappable element 972 of the flow indicators 970. In contrast, if fluid is not flowing through the first channel 104a (and thus not flowing through the bypass channel portion 964), the flappable elements 972 will remain generally stationary. Accordingly, movement of the flow indicators 970 can be detected by a physician or other healthcare provider to confirm fluid is flowing through the first channel 104a.

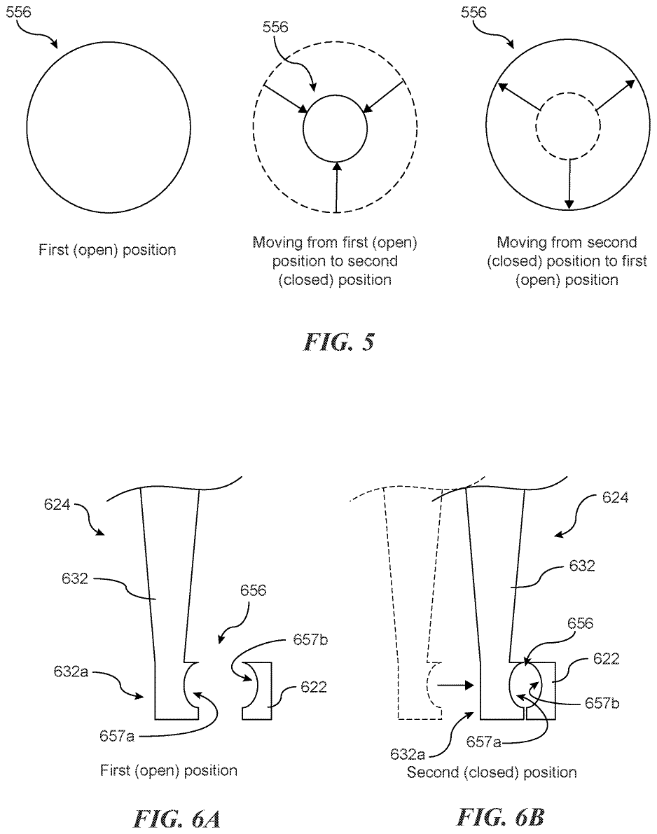

FIG. 10 illustrates a fourth embodiment of a flow indicator assembly 1060 for detecting fluid flow through a shunt and configured in accordance with select embodiments of the present technology. Similar to the flow indicator assemblies 540 and 640 of FIGS. 5 and 6, respectively, the flow indicator assembly 1060 includes a bypass channel portion 1064 extending from and fluidly coupled to the first channel 104a. The flow indicator assembly 1060 further includes a flow indicator 1070 positioned within the bypass channel portion 1064. The flow indicator 1070 includes a flappable element 1072 (e.g., a sheet of fabric or other moveable element) fixedly coupled to a side of the bypass channel portion 1064 via a connector 1074 (e.g., a rod). Relative to the flappable element 972 of the flow indicators 970 in FIG. 9, the flappable element 1072 of the flow indicator 1070 is larger and configured to occupy a greater area of the bypass channel portion 1064. That is, the flappable element 1072 can be a sheet of fabric (e.g., a flag-like structure) instead of a thread-like structure. The flow indicator assembly 1060 operates generally similarly to the flow indicator assembly 960 of FIG. 9. In particular, as fluid (shown using broken-line arrows) flows through the first channel 104a and thus the bypass channel portion 1064, the fluid agitates or otherwise induces motion in the flappable element 1072 of the flow indicator 1070. In contrast, if fluid is not flowing through the first channel 104a (and thus not flowing through the bypass channel portion 1064), the flappable element 1072 remains generally stationary. Accordingly, movement of the flow indicator 1070 can be detected by a physician or other healthcare provider to confirm fluid is flowing through the first channel 104a.

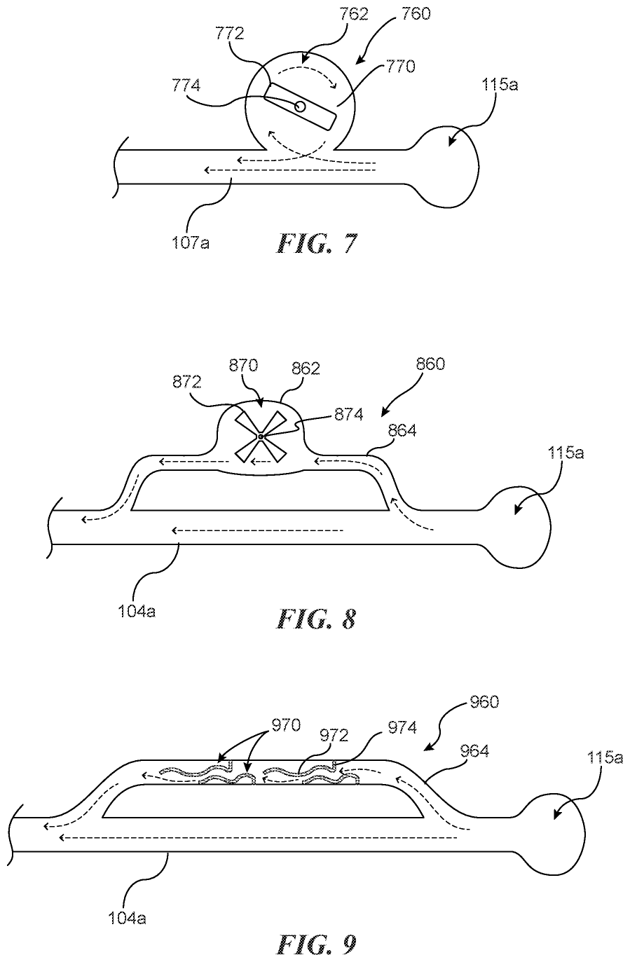

FIG. 11 illustrates a fifth embodiment of a flow indicator assembly 1160 for detecting fluid flow through a shunt and configured in accordance with select embodiments of the present technology. Similar to previously described embodiments, the flow indicator assembly 1160 includes a bypass channel portion 1164 extending from and fluidly coupled to the first channel 104a. The flow indicator assembly 1160 further includes a flow indicator 1170 comprising a plurality of unconstrained elements 1172 positioned between two gates 1174. The unconstrained elements 1172, which can have spherical or bead-like shapes, are not directly coupled to any side of the bypass channel portion 1164. Instead, the gates 1174 prevent the unconstrained elements 1172 from flowing out of the bypass channel portion 1164 while simultaneously permitting fluid to pass through the bypass channel portion 1164. For example, the gates 1174 may be composed of a mesh or other substance having a plurality of holes that are smaller than the unconstrained elements 1172 such that fluid can flow through the holes but the unconstrained elements 1172 cannot. This is expected to prevent the unconstrained elements 1172 from flowing out of the bypass channel portion 846. In operation, some of the fluid (shown as broken-line arrows) flowing through the first channel 104a will flow into the bypass channel portion 1164 and through the gates 1174. As the fluid flows through the bypass channel portion 1164, the fluid will agitate or otherwise induce motion in the unconstrained elements 1172. In contrast, if fluid is not flowing through the first channel 104a (and thus not flowing through the bypass channel portion 1164), the unconstrained elements 1172 will remain generally stationary. Accordingly, movement of the unconstrained elements 1172 can be detected by a physician or other healthcare provider to confirm fluid is flowing through the first channel 104a.

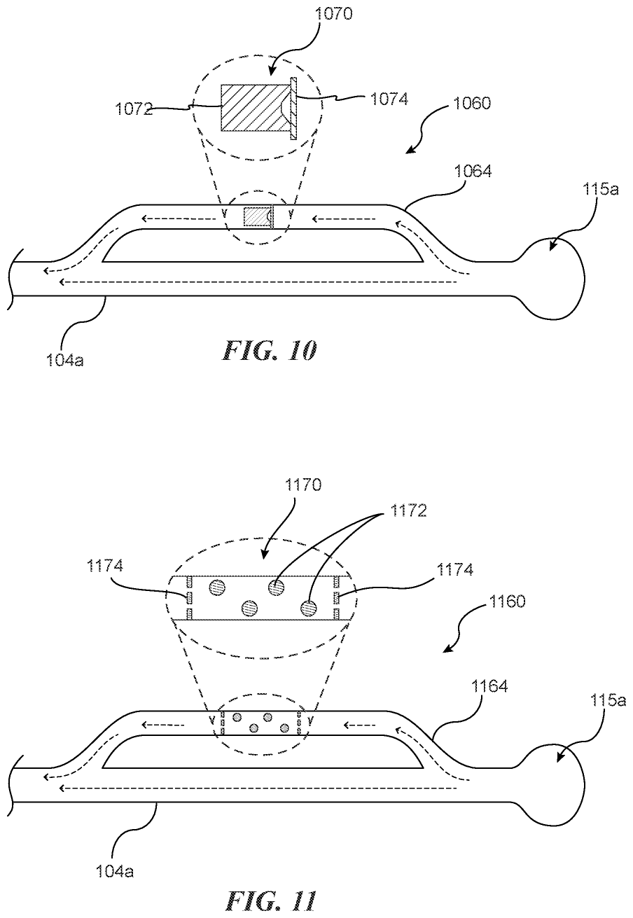

FIG. 12 illustrates a sixth embodiment of a flow indicator assembly 1260 for detecting fluid flow through a shunt and configured in accordance with select embodiments of the present technology. As shown, the flow indicator assembly 1260 includes a flow indicator 1270 having an annular flow path 1272 positioned generally between the first well 115a and the first channel 104a. The annular flow path 1272 of the flow indicator 1270 can have a cross-sectional area that is smaller than (e.g., less than 50% of) the cross-sectional area of the first well 115a. Thus, as fluid (shown as broken-line arrows) flows from the first well 115a into the annular flow path 1272, the fluid will accelerate in velocity and experience a decrease in static pressure (e.g., demonstrating a Venturi effect). As a result, one or more bubbles will be formed in the fluid, which can be visualized flowing through the annular flow path 1272. In such embodiments, the presence of bubbles in the annular flow path 1272 indicates fluid is flowing through the flow indicator assembly 1260 and into the first channel 104a, whereas the absence of bubbles in the annular flow path 1272 indicates fluid is not flowing through the flow indicator assembly 1260 and thus is not flowing into or through the first channel 104a.

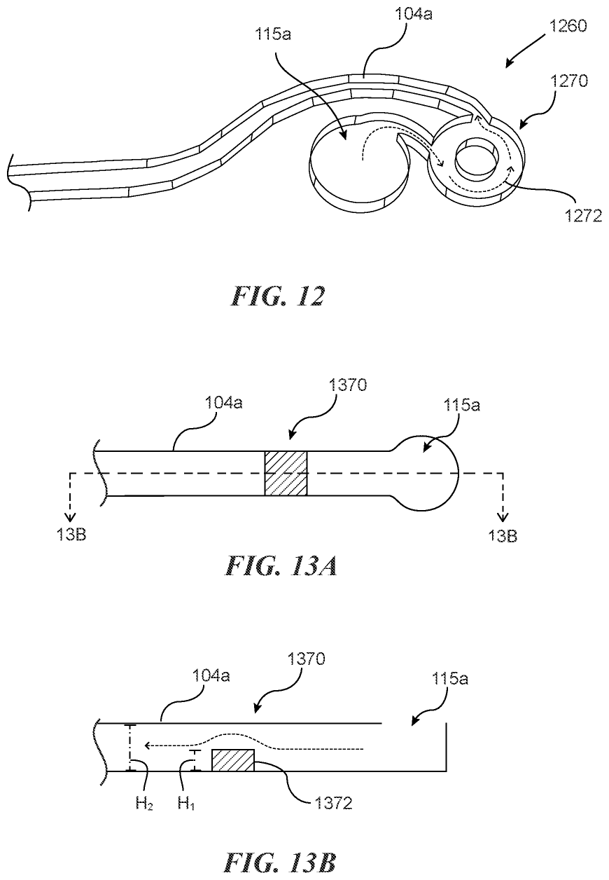

FIGS. 13A and 13B illustrate a seventh embodiment of a flow indicator 1370 for detecting fluid flow through a shunt and configured in accordance with select embodiments of the present technology. In particular, FIG. 13A is a top view of the flow indicator 1370, and FIG. 13B is a side cross-sectional view of the flow indicator 1370 taken along the lines indicated in FIG. 13A. As best shown in FIG. 13B, the flow indicator 1370 includes a protrusion or bump 1372 that partially obstructs flow through the first channel 104a. The protrusion 1372 can have a first height H₁ that is less than a corresponding second height H₂ of the channel. In some embodiments, the first height H₁ may be between 20% and 70% of the second height H₂. Fluid (shown as a broken-line arrow) flowing through the first channel 104a is a least partially obstructed by the protrusion 1372. As a result, the flow is at least partially disrupted, which may generate visible bubbles in the fluid. Accordingly, the presence of bubbles adjacent the flow indicator 1370 indicates fluid is flowing through the first channel 104a, whereas the absence of bubbles in the first channel 104a may indicate that fluid is not flowing through the first channel 104a.

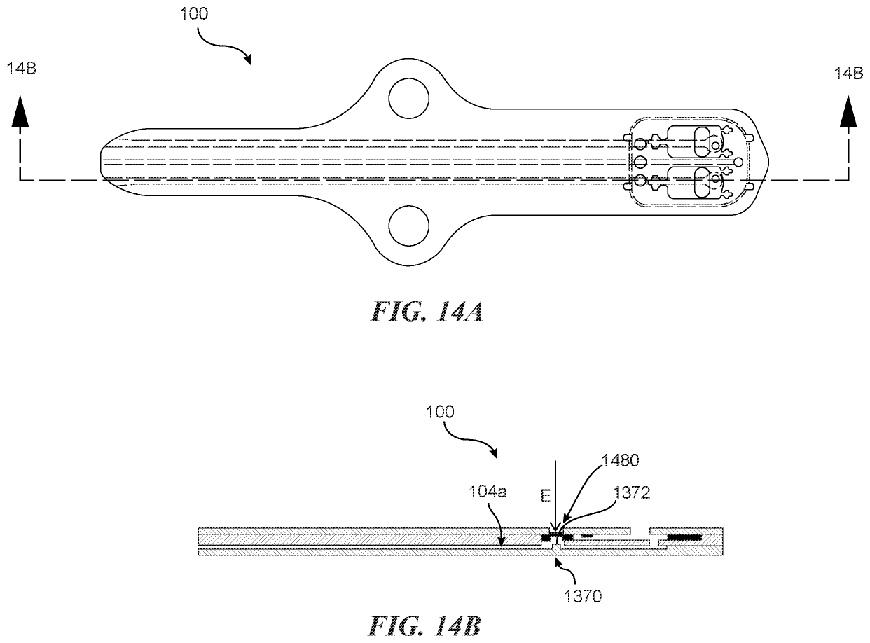

In some embodiments, external energy can be directed at the flow indicator 1370 to increase the number of bubbles formed proximate the flow indicator 1370. For example, FIG. 14A illustrates the system 100, and FIG. 14B is a cross sectional illustration of the system taken along the line labeled 14B-14B in FIG. 14A. As shown in FIG. 14B, the system 100 can include a window 1480 positioned generally above/vertically aligned with the flow indicator 1370 and extending through the system 100. Energy (e.g., laser energy) E can be directed at the protrusion 1372 of the flow indicator 1370 through the window 1480. The energy E can disturb fluid flowing past the flow indicator 1370, thus causing additional bubbles to form. In some embodiments, generating bubbles using an external energy input such as a laser may be advantageous because more bubbles may be generated compared to embodiments without the external energy input, which is expected to make it easier for a physician to quickly verify the presence of flow through the first channel 104a.

Although many of the flow indicators shown and described herein are positioned in bypass channels or eddies off a primary flow channel/lumen (e.g., the first channel 104a), in some embodiments the flow indicators can be positioned in the primary flow channel itself. For example, any of the flow indicators described with reference to FIGS. 7-14B can be positioned directly in the first channel 104a, such as described with reference to the flow indicator 1370 in FIGS. 13A-14B. However, in some embodiments it is expected to be advantageous to position the flow indicators in a bypass channel or other offshoot from the primary flow channel. In such embodiments, flow through the primary flow channel would not be interrupted if the flow indicator inadvertently or temporarily blocks flow.

The present technology can include additional flow indicators that can be used in combination with, or in lieu of, those described with reference to FIGS. 7-14B. For example, in some embodiments a flow indicator assembly can include a reservoir housing a transient flow indicator such as fluorescein. The reservoir can be activated (e.g., via external energy input) to selectively release the fluorescein. If flow is occurring through the shunt when the fluorescein is released, the fluorescein will flow through the shunt. If flow is not occurring through the shunt when the fluorescein is released, the fluorescein will pool adjacent the reservoir.

In some embodiments, the flow indicator assemblies and flow indicators described herein can be positioned within a portion of the system 100 that is expected to be generally visible after the system 100 is implanted in the patient. For example, referring back to FIG. 1A, the flow indicator assemblies and flow indicators may be positioned along the first channel 104a in a section of the shunting element 102 that is downstream of the plate 122. That way, the flow indicator assembly and/or flow indicator are not blocked by the plate 122. In other embodiments, and similar to the arrangement described with reference to FIGS. 14A and 14B, the system 100 may include a window, mirror, or other feature that aids with the visualization of the flow indicator assembly and/or flow indicator.

As set forth above, any of the flow indicator assemblies and flow indicators described herein can be used to determine flow through the first channel 104a of the system 100 and/or through another channel of the system 100. Likewise, the flow indicator assemblies and flow indicators described herein can be used to determine flow through other adjustable shunts, such as the adjustable shunting system of FIGS. 3A and 3B, and any of the adjustable shunting systems incorporated by reference in this application. Further yet, the flow indicator assemblies and flow indicators described herein can be used to determine flow through non-adjustable shunts. Accordingly, the present technology is not limited to the particular configurations shown herein.

### D. Additional Embodiments of Shunting Systems

In some embodiments, shunting systems of the present technology can include both system state indicators and flow indicators. For example, a shunting system (e.g., the system 100 or the system 300) may include both the state indicators described with reference to FIGS. 2A-6B, and one or more of the flow indicators described with reference to FIGS. 7-14B. Without intending to be bound by theory, including both a state indicator and a flow indicator is expected to be useful because it enables a physician to confirm that a system is operating as intended.

The systems described herein can be designed for shunting fluid between a variety of body regions. As noted above, for example, in some embodiments the systems described herein are designed to be implanted in a patient's eye to shunt aqueous between the anterior chamber and a target outflow location (e.g., a subconjunctival bleb space), such as to treat glaucoma. Accordingly, in some embodiments the systems described herein can have dimensions compatible with being implanted in the patient's eye. For example, the systems described herein (e.g., the system 100) may have a length of between about 4 mm and about 20 mm, such as between about 4 mm and 15 mm, or between about 4 mm and 12 mm, or between about 6 mm and 10 mm, or about 8 mm. In some embodiments, the layers (e.g., the first layer 110, the second layer 112, and/or third layer 114) can have a width or thickness less than about 500 microns, less than about 400 microns, less than about 300 microns, and/or less than about 200 microns. In some embodiments, the diameter of the fluidic channels and corresponding apertures (e.g., the channels 104) may be less than about 100 microns, less than about 75 microns, and/or less than about 50 microns, such as about 35 microns. The foregoing dimensions are provided by way of example only, and other dimensions outside the ranges provided above are possible and included within the scope of the present technology. Indeed, the dimensions of the systems described herein may be designed depending on the type of shunting system (e.g., glaucoma shunt vs. hydrocephalus shunt) and intended recipient (e.g., child vs. adult).

### Examples

Several aspects of the present technology are set forth in the following examples:

1. An adjustable shunting system for shunting fluid from a first body region to a second body region within a patient, the system comprising:
a shunting element having a channel extending therethrough, wherein, when the system is implanted in the patient, the shunting element is configured to extend between the first body region and the second body region;
an actuator for selectively controlling the flow of fluid through the shunting element, wherein the actuator includes a gating element that is transitionable between at least a first position associated with a first fluid resistance through the shunting element and a second position associated with a second fluid resistance through the shunting element;
an actuator position indicator for determining whether the gating element is in the first position or the second position; and
one or more adjustment indicators, including at least one of—
   a first adjustment indicator identifying a first actuation element for transitioning the gating element from the first position to and/or toward the second position; or
   a second adjustment indicator identifying a second actuation element for transitioning the gating element from the second position to and/or toward the first position.

2. The system of example 1, further comprising a plate coupled to the actuator, wherein the plate includes the actuator position indicator and the one or more adjustment indicators.

3. The system of example 2 wherein the plate is composed of a non-transparent material.

4. The system of example 2 or example 3 wherein the actuator position indicator includes one or more holes extending through the plate.

5. The system of example 4 wherein the one or more holes includes a plurality of holes having different diameters.

6. The system of example 2 wherein the actuation position indicator includes an opening with a marker.

7. The system of example 6 wherein the marker forms part of a perimeter of the opening.

8. The system of example 6 or example 7 wherein the marker is a tooth, tab, projection, notch, and/or groove.

9. The system of example 6 wherein the marker includes one or more bridge elements extending across the opening.

10. The system of example 2 wherein the actuator position indicator includes a first opening portion and a second opening portion.

11. The system of example 10 wherein the first opening portion and the second opening portion have different shapes.

12. The system of example 10 wherein the first opening portion and the second opening portion are not connected.

13. The system of example 10 wherein one of the first opening portion or the second opening portion is composed of a plurality of smaller, discrete openings.

14. The system of any of examples 2-13 wherein the first adjustment indicator and/or the second adjustment indicator have a different color than the plate.

15. The system of any of examples 1-14 wherein the system includes both the first adjustment indicator and the second adjustment indicator.

16. The system of example 15 wherein the plate includes a first opening aligned with the first actuation element and a second opening aligned with the second actuation element, and wherein the first adjustment indicator is positioned directly adjacent the first opening and the second adjustment indicator is positioned directly adjacent the second opening.

17. The system of example 15 or example 16 wherein the first adjustment indicator is a decrease flow indicator and the second adjustment indicator is an increase flow indicator.

18. The system of any of examples 15-17 wherein the first adjustment indicator has a dash shape, and wherein the second adjustment indicator has a chevron shape or a plus shape.

19. The system of any of examples 1-14 wherein the system includes only one of the first adjustment indicator or the second adjustment indicator.

20. The adjustable shunting system of any of examples 1-19 wherein the system is an intraocular shunting system.

21. An adjustable shunting system for shunting fluid from a first body region to a second body region within a patient, the system comprising:
a shunting element having a channel extending therethrough, wherein, when the system is implanted in the patient, the shunting element is configured to extend between the first body region and the second body region;
an actuator for selectively controlling the flow of fluid through the shunting element, wherein the actuator includes a gating element that is transitionable between at least a first position associated with a first fluid resistance through the shunting element and a second position associated with a second fluid resistance through the shunting element; and
a plate coupled to the actuator, the plate including an actuator position indicator for indicating whether the gating element is in the first position or the second position.

22. The system of example 21 wherein the actuator position indicator includes one or more holes extending through the plate.

23. The system of example 22 wherein the one or more holes includes a plurality of holes having different diameters.

24. The system of example 21 wherein the actuation position indicator includes an opening with a marker.

25. The system of example 24 wherein the marker forms part of a perimeter of the opening.

26. The system of example 24 or example 25 wherein the marker is a tooth, tab, projection, notch, and/or groove.

27. The system of example 24 wherein the marker includes one or more bridge elements extending across the opening.

28. The system of example 21 wherein the actuator position indicator includes a first opening portion and a second opening portion.

29. The system of example 28 wherein the first opening portion and the second opening portion have different shapes.

30. The system of example 28 wherein the first opening portion and the second opening portion are not connected.

31. The system of example 28 wherein one of the first opening portion or the second opening portion is composed of a plurality of smaller, discrete openings.

32. An adjustable shunting system for shunting fluid from a first body region to a second body region within a patient, the system comprising:
a shunting element having a channel extending therethrough, wherein, when the system is implanted in the patient, the shunting element is configured to extend between the first body region and the second body region;
an actuator for selectively controlling the flow of fluid through the shunting element, wherein the actuator includes a gating element that is transitionable between at least a first position associated with a first fluid resistance through the shunting element and a second position associated with a second fluid resistance through the shunting element; and
a plate coupled to the actuator, the plate including one or more adjustment indicators, including at least one of—
   a first adjustment indicator indicating a first actuation element for transitioning the gating element from the first position to and/or toward the second position; or
   a second adjustment indicator indicating a second actuation element for transitioning the gating element from the second position to and/or toward the first position.

33. The system of example 32 wherein the first adjustment indicator and/or the second adjustment indicator have a different color than the plate.

34. The system of example 32 or example 33 wherein the system includes both the first adjustment indicator and the second adjustment indicator.

35. The system of example 34 wherein the plate includes a first opening aligned with the first actuation element and a second opening aligned with the second actuation element, and wherein the first adjustment indicator is positioned directly adjacent the first opening and the second adjustment indicator is positioned directly adjacent the second opening.

36. The system of example 34 or example 35 wherein the first adjustment indicator is a decrease flow indicator and the second adjustment indicator is an increase flow indicator.

37. The system of any of examples 34-36 wherein the first adjustment indicator has a dash shape, and wherein the second adjustment indicator has a chevron shape or a plus shape.

38. The system of example 32 or example 33 wherein the system includes only one of the first adjustment indicator or the second adjustment indicator.

39. A shunting system for shunting fluid from a first body region to a second body region, the shunting system comprising:
a shunting element having a channel extending therethrough, wherein, when the system is implanted in the patient, the shunting element is configured to extend between the first body region and the second body region; and
a flow indicator assembly coupled to the channel and configured to provide visual feedback to confirm whether fluid is flowing through the channel.

40. The shunting system of example 39 wherein the flow indicator assembly includes a flow indicator and a bypass channel portion fluidly coupled to the channel, and wherein the flow indicator is positioned within the bypass channel portion.

41. The shunting system of example 39 wherein the flow indicator assembly includes a flow indicator and an eddy fluidly coupled to the channel, and wherein the flow indicator is positioned within the eddy.

42. The shunting system of example 39 wherein the flow indicator assembly includes a flow indicator, and wherein the flow indicator is positioned within the channel.

43. The shunting system of any of examples 39-42 wherein the flow indicator includes a fan rotatably coupled to a wall of the channel, and wherein the fan is configured to rotate when fluid is flowing through the channel.

44. The shunting system of any of examples 39-42 wherein the flow indicator includes a flappable element coupled to a wall of the channel, and wherein the flappable element is configured to move when fluid is flowing through the channel.

45. The shunting system of any of examples 39-42 wherein the flow indicator includes one or more unconstrained elements positioned between two gates, and wherein the one or more unconstrained elements are configured to move between the two gates when fluid is flowing through the channel.

46. The shunting system of any of examples 39-42 wherein the flow indicator includes an annular flow path, and wherein, when fluid flows through the annular flow path, bubbles form in the fluid.

47. The shunting system of any of examples 39-42 wherein the flow indicator includes a protrusion, and wherein, when fluid flows through the channel, bubbles form in the fluid.

48. The shunting system of any of examples 39-47 wherein the shunting system is an adjustable shunting system.

49. The shunting system of any of examples 39-47 wherein the shunting system is a non-adjustable shunting system.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An adjustable shunting system for shunting fluid from a first body region to a second body region within a patient, the system comprising:
a shunting element having a channel extending therethrough, wherein, when the system is implanted in the patient, the shunting element is configured to extend between the first body region and the second body region;
an actuator for selectively controlling the flow of fluid through the shunting element, wherein the actuator includes a gating element that is transitionable between at least a first position associated with a first fluid resistance through the shunting element and a second position associated with a second fluid resistance through the shunting element;
an actuator position indicator for determining whether the gating element is in the first position or the second position; and
one or more adjustment indicators, including at least one of—
a first adjustment indicator identifying a first actuation element for transitioning the gating element from the first position to and/or toward the second position; or
a second adjustment indicator identifying a second actuation element for transitioning the gating element from the second position to and/or toward the first position.

2. The system of claim 1, further comprising a plate coupled to the actuator, wherein the plate includes the actuator position indicator and the one or more adjustment indicators.

3. The system of claim 2 wherein the plate is composed of a non-transparent material.

4. The system of claim 2 wherein the actuator position indicator includes one or more holes extending through the plate.

5. The system of claim 4 wherein the one or more holes includes a plurality of holes having different diameters.

6. The system of claim 2 wherein the actuation position indicator includes an opening with a marker.

7. The system of claim 6 wherein the marker forms part of a perimeter of the opening.

8. The system of claim 6 wherein the marker is a tooth, tab, projection, notch, and/or groove.

9. The system of claim 6 wherein the marker includes one or more bridge elements extending across the opening.

10. The system of claim 2 wherein the actuator position indicator includes a first opening portion and a second opening portion.

11. The system of claim 10 wherein the first opening portion and the second opening portion have different shapes.

12. The system of claim 10 wherein the first opening portion and the second opening portion are not connected.

13. The system of claim 10 wherein one of the first opening portion or the second opening portion is composed of a plurality of smaller, discrete openings.

14. The system of claim 2 wherein the first adjustment indicator and/or the second adjustment indicator have a different color than the plate.

15. The system of claim 1 wherein the system includes both the first adjustment indicator and the second adjustment indicator.

16. The system of claim 15 wherein the plate includes a first opening aligned with the first actuation element and a second opening aligned with the second actuation element, and wherein the first adjustment indicator is positioned directly adjacent the first opening and the second adjustment indicator is positioned directly adjacent the second opening.

17. The system of claim 15 wherein the first adjustment indicator is a decrease flow indicator and the second adjustment indicator is an increase flow indicator.

18. The system of claim 15 wherein the first adjustment indicator has a dash shape, and wherein the second adjustment indicator has a chevron shape or a plus shape.

19. The system of claim 1 wherein the system includes only one of the first adjustment indicator or the second adjustment indicator.

20. The adjustable shunting system of claim 1 wherein the system is an intraocular shunting system.

21. An adjustable shunting system for shunting fluid from a first body region to a second body region within a patient, the system comprising:
a shunting element having a channel extending therethrough, wherein, when the system is implanted in the patient, the shunting element is configured to extend between the first body region and the second body region;
an actuator for selectively controlling the flow of fluid through the shunting element, wherein the actuator includes a gating element that is transitionable between at least a first position associated with a first fluid resistance through the shunting element and a second position associated with a second fluid resistance through the shunting element; and
a plate coupled to the actuator, the plate including an actuator position indicator for indicating whether the gating element is in the first position or the second position.

22. The system of claim 21 wherein the actuator position indicator includes one or more holes extending through the plate.

23. The system of claim 22 wherein the one or more holes includes a plurality of holes having different diameters.

24. The system of claim 21 wherein the actuation position indicator includes an opening with a marker.

25. The system of claim 24 wherein the marker forms part of a perimeter of the opening.

26. The system of claim 24 wherein the marker is a tooth, tab, projection, notch, and/or groove.

27. The system of claim 24 wherein the marker includes one or more bridge elements extending across the opening.

28. The system of claim 21 wherein the actuator position indicator includes a first opening portion and a second opening portion.

29. The system of claim 28 wherein the first opening portion and the second opening portion have different shapes.

30. The system of claim 28 wherein the first opening portion and the second opening portion are not connected.

31. The system of claim 28 wherein one of the first opening portion or the second opening portion is composed of a plurality of smaller, discrete openings.

32. An adjustable shunting system for shunting fluid from a first body region to a second body region within a patient, the system comprising:
a shunting element having a channel extending therethrough, wherein, when the system is implanted in the patient, the shunting element is configured to extend between the first body region and the second body region;
an actuator for selectively controlling the flow of fluid through the shunting element, wherein the actuator includes a gating element that is transitionable between at least a first position associated with a first fluid resistance through the shunting element and a second position associated with a second fluid resistance through the shunting element; and
a plate coupled to the actuator, the plate including one or more adjustment indicators, including at least one of—
a first adjustment indicator indicating a first actuation element for transitioning the gating element from the first position to and/or toward the second position; or
a second adjustment indicator indicating a second actuation element for transitioning the gating element from the second position to and/or toward the first position.

33. The system of claim 32 wherein the first adjustment indicator and/or the second adjustment indicator have a different color than the plate.

34. The system of claim 32 wherein the system includes both the first adjustment indicator and the second adjustment indicator.

35. The system of claim 34 wherein the plate includes a first opening aligned with the first actuation element and a second opening aligned with the second actuation element, and wherein the first adjustment indicator is positioned directly adjacent the first opening and the second adjustment indicator is positioned directly adjacent the second opening.

36. The system of claim 34 wherein the first adjustment indicator is a decrease flow indicator and the second adjustment indicator is an increase flow indicator.

37. The system of claim 34 wherein the first adjustment indicator has a dash shape, and wherein the second adjustment indicator has a chevron shape or a plus shape.

38. The system of claim 32 wherein the system includes only one of the first adjustment indicator or the second adjustment indicator.

39. A shunting system for shunting fluid from a first body region to a second body region, the shunting system comprising:
a shunting element having a channel extending therethrough, wherein, when the system is implanted in the patient, the shunting element is configured to extend between the first body region and the second body region; and
a flow indicator assembly coupled to the channel and configured to provide visual feedback to confirm whether fluid is flowing through the channel.

40. The shunting system of claim 39 wherein the flow indicator assembly includes a flow indicator and a bypass channel portion fluidly coupled to the channel, and wherein the flow indicator is positioned within the bypass channel portion.

41. The shunting system of claim 39 wherein the flow indicator assembly includes a flow indicator and an eddy fluidly coupled to the channel, and wherein the flow indicator is positioned within the eddy.

42. The shunting system of claim 39 wherein the flow indicator assembly includes a flow indicator, and wherein the flow indicator is positioned within the channel.

43. The shunting system of claim 39 wherein the flow indicator includes a fan rotatably coupled to a wall of the channel, and wherein the fan is configured to rotate when fluid is flowing through the channel.

44. The shunting system of claim 39 wherein the flow indicator includes a flappable element coupled to a wall of the channel, and wherein the flappable element is configured to move when fluid is flowing through the channel.

45. The shunting system of claim 39 wherein the flow indicator includes one or more unconstrained elements positioned between two gates, and wherein the one or more unconstrained elements are configured to move between the two gates when fluid is flowing through the channel.

46. The shunting system of claim 39 wherein the flow indicator includes an annular flow path, and wherein, when fluid flows through the annular flow path, bubbles form in the fluid.

47. The shunting system of claim 39 wherein the flow indicator includes a protrusion, and wherein, when fluid flows through the channel, bubbles form in the fluid.

48. The shunting system of claim 39 wherein the shunting system is an adjustable shunting system.

49. The shunting system of claim 39 wherein the shunting system is a non-adjustable shunting system.
